(19) United States
(12) Patent Application Publication
(10) Pub. No.: US 2003/0039647 A1
(43) Pub. Date: Feb. 27, 2003

(54) Tetraspan protein and uses thereof

(76) Inventors: Christoph Reinhard, Alameda, CA (US); Pablo D. Garcia, San Francisco, CA (US)

Correspondence Address: Chiron Corporation, Intellectual Property R338, P.O. Box 8097, Emeryville, CA, 94662-8097, US

(21) Appl. No.: 09/905,674

(22) Filed: Jul. 13, 2001

Related U.S. Application Data
(60) Provisional application No. 60/218,280, filed on Jul. 14, 2000.

(51) Int. Cl. A61K 48/00; A61K 39/395; C07H 21/04; C07K 16/42

(52) U.S. Cl. 424/141.1; 514/44; 530/388.15; 536/23.2

(57) ABSTRACT

Inhibitors of TSPAN-7 are provided that reduce the expression or biological activities of TSPAN-7 in a mammalian cell. TSPAN-7 inhibitors include antisense molecules, ribozymes, antibodies and antibody fragments, proteins and polypeptides as well as small molecules. TSPAN-7 inhibitors find use in compositions and methods for decreasing TSPAN-7 gene expression as well as methods for inhibiting proliferation of mammalian cells, including tumor cells, methods for decreasing the side effects of cancer therapy and methods for treating neoplastic diseases. Also provided are nucleic acids encoding a new member of the tetraspan family, TSPAN-7, and polypeptides encoded thereby.

### CROSS-REFERENCE TO RELATED APPLICATION

This application claims the benefit of U.S. Provisional Patent Application No. 60/218,280 filed Jul. 14, 2000, where this provisional application is incorporated herein by reference in its entirety.

### BACKGROUND OF THE INVENTION

1. Technical Field

The present invention provides a new tetraspan protein, polynucleotides encoding the protein, and compositions and methods for inhibiting tetraspan protein expression and/or biological activity. Such compositions and methods find utility in the treatment of neoplastic disease.

2. Description of the Related Art

The tetraspan family is discussed in Maecker, H.T. et al., FASEB J. 11:428-442, 1997. Expression of tetraspan genes in lymphoma cell lines is discussed in Ferrer, M. et al., Clin. Exp. Immunol. 113:346-352, 1998.

### SUMMARY OF THE INVENTION

The present invention provides, in one embodiment, a novel tetraspan protein encoded by SEQ ID NO:1, and referred to as TSPAN-7 (SEQ ID NO:2).

The invention further provides an isolated nucleic acid molecule comprising a polynucleotide selected from the group consisting of:

(a) a polynucleotide encoding amino acids from about 1 to about 270 of SEQ ID NO:2;

(b) a polynucleotide encoding amino acids from about 2 to about 270 of SEQ ID NO:2;

(c) the polynucleotide complement of the polynucleotide of (a) or (b); and

(d) a polynucleotide at least 90% identical to the polynucleotide of (a), (b), or (c).

The invention still further provides an isolated nucleic acid molecule comprising at least 810 contiguous nucleotides from the coding region of SEQ ID NO:1.

The invention also provides an isolated nucleic acid molecule comprising a polynucleotide encoding a polypeptide wherein, except for at least one conservative amino acid substitution, the polypeptide has an amino acid sequence selected from the group consisting of:

(a) a polynucleotide encoding amino acids from about 1 to about 270 of SEQ ID NO:2;

(b) a polynucleotide encoding amino acids from about 2 to about 270 of SEQ ID NO:2;

(c) the polynucleotide complement of the polynucleotide of (a) or (b); and

(d) a polynucleotide at least 90% identical to the polynucleotide of (a), (b), or(c).

The invention further provides an isolated nucleic acid molecule having the sequence of SEQ ID NO:1, wherein the nucleic acid molecule is DNA.

In a further embodiment the invention provides a method of making a recombinant vector comprising inserting a nucleic acid molecule of any one of SEQ ID NO:1 and 3-7 into a vector in operable linkage to a promoter, a recombinant vector produced by this method, and a method of making a recombinant host cell comprising introducing the recombinant vector into a host cell.

The invention further provides an isolated polypeptide comprising amino acids at least 95% identical to a polypeptide comprising amino acids from about 1 to about 270 of SEQ ID NO:2, and an isolated polypeptide wherein, except for at least one conservative amino acid substitution, the polypeptide has an amino acid sequence selected from the group consisting of:

(a) a polynucleotide encoding amino acids from about 1 to about 270 of SEQ ID NO:2;

(b) a polynucleotide encoding amino acids from about 2 to about 270 of SEQ ID NO:2;

(c) the polynucleotide complement of the polynucleotide of (a) or (b): and

(d) a polynucleotide at least 90% identical to the polynucleotide of (a), (b), or (c).

The invention also provides a portion of the TSPAN-7 protein, comprising SEQ ID NO:13 or SEQ ID NO:14, and fusion proteins comprising at least one of SEQ ID NO:13 and 14, or a fragment thereof.

The invention still further provides an epitope-bearing portion of the polypeptide of SEQ ID NO:2; in one embodiment, the epitope-bearing portion comprises about 8 to about 25 contiguous amino acids of SEQ ID NO:2, more preferably about 10 to about 15 contiguous amino acids of SEQ ID NO:2.

The invention also provides an isolated antibody that binds specifically to a polypeptide of SEQ ID NO:2, or a portion thereof, wherein the antibody may be a polyclonal antibody, a monoclonal antibody, a humanized antibody, or an antibody fragment.

The invention further provides an isolated TSPAN-7 inhibitor wherein said TSPAN-7 inhibitor is an antisense molecule; in one embodiment the antisense molecule or the complement thereof comprises at least 10 consecutive nucleic acids of the sequence of SEQ ID NO:1; and in another embodiment the antisense molecule or the complement thereof hybridizes under high stringency conditions to the sequence of SEQ ID NO:1.

The invention still further provides an antisense molecule comprising a nucleic acid sequence selected from the group consisting of SEQ ID NO:3-7.

The invention also provides an isolated TSPAN-7 inhibitor wherein the TSPAN-7 inhibitor is a ribozyme. In other embodiments the TSPAN-7 inhibitor is selected from the group consisting of an antibody and an antibody fragment; and the antibody or antibody fragment may be monoclonal, and the antibody or antibody fragment may be humanized.

The invention still further provides a composition comprising a therapeutically effective amount of a TSPAN-7 inhibitor in a pharmaceutically acceptable carrier; in certain embodiments the composition may comprise two ore more TSPAN-7 inhibitors, and in one embodiment the TSPAN-7 inhibitor is an antisense molecule.

The invention also provides a method of decreasing the expression of TSPAN-7 in a mammalian cell, comprising administering to the cell a TSPAN-7 inhibitor, wherein the TSPAN-7 inhibitor is an antisense molecule, a ribozyme, an antibody, an antibody fragment, a protein, a polypeptide, or a small molecule.

The invention further provides a method of treating a hyperproliferative disorder comprising administering to a mammalian cell a TSPAN-7 inhibitor such that the hyperproliferative disorder is reduced in severity. In certain embodiments the hyperproliferative disorder is cancer.

### BRIEF DESCRIPTION OF THE FIGURES

FIG. 1 is a schematic showing the structure of tetraspan proteins. Amino acid (N) and carboxyl (C) termini and extracellular and transmembrane domains are indicated. Highly conserved amino acids, found in at least 12 of 18 tetraspan genes, are shown in circles. Highly conserved amino acids found in 14 or more tetraspans are shown in boldface circles. The conserved PXSC motif is located a different positions within EC2 in the various tetraspans, and is therefore indicated with floating arrows. Asterisks indicate conserved charged amino acids within the transmembrane domains. (Maecker et al., FASEB J. 11:428-442, 1997.)

FIG. 2 (A, B and C) is a polynucleotide sequence of 1387 base pairs (SEQ ID NO:1) which encodes TSPAN-7.

FIG. 3 shows the TSPAN-7 amino acid sequence (SEQ ID NO:2) of 270 amino acids encoded by SEQ ID NO: 1.

FIG. 4 provides antisense and control (RC) oligonucleotides (SEQ ID NO:3-12) based on SEQ ID NO:1.

FIG. 5 is a bar graph showing the effect of antisense and control oligonucleotides on TSPAN-7 mRNA levels normalized to actin mRNA in SW620 cells.

FIG. 6 is a graph showing the effect of antisense oligonucleotide of SEQ ID NO:6 (22-4AS) and control oligonucleotide of SEQ ID NO:11 (22-4RC) on mRNA levels in SW620 cells over 4 days of growth.

### DETAILED DESCRIPTION OF THE INVENTION

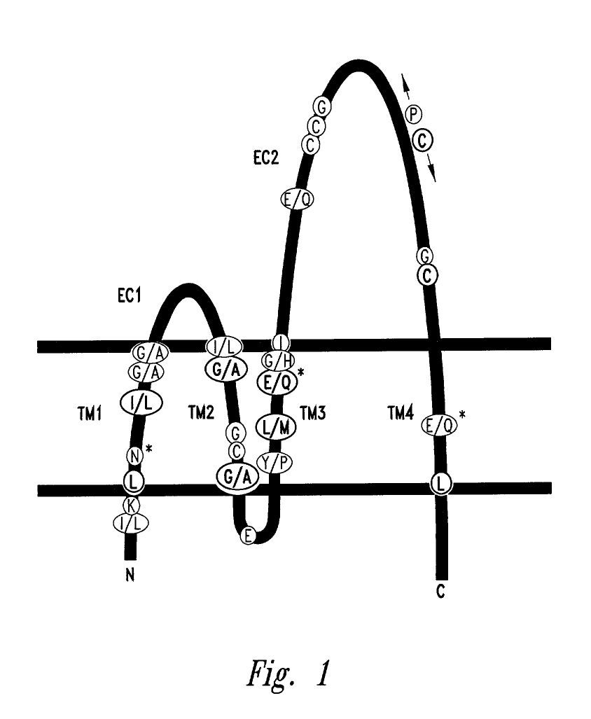

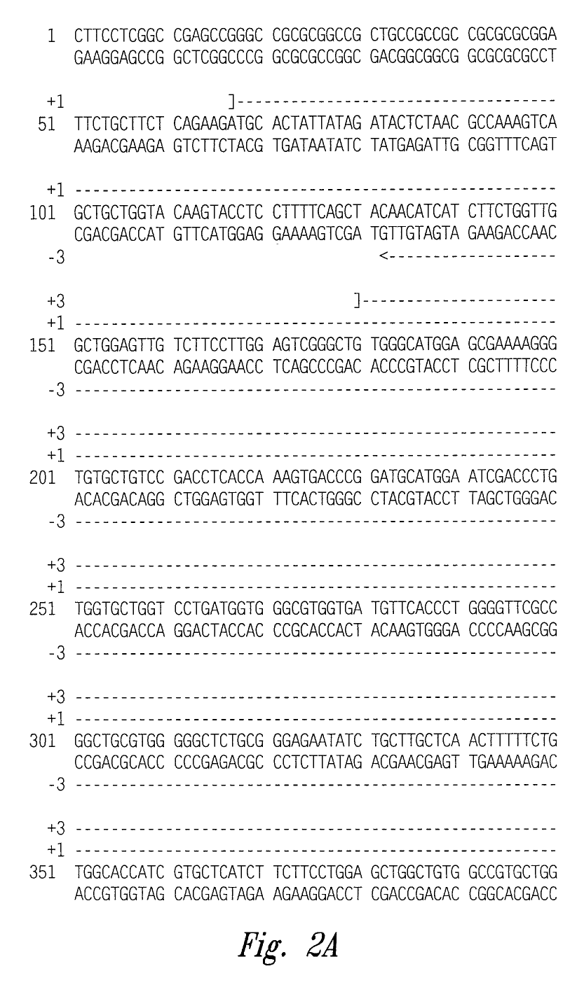

Proteins of the tetraspan superfamily are characterized by four transmembrane domains and two extracellular regions; a schematic diagram of a four transmembrane protein is shown in FIG. 1. Within the superfamily is a specific family referred to as NET proteins, for “new EST tetraspan” (Serru, V. et al., Biochem. Biophys. Acta 1478:159-163, 2000). Serru et al. reported the existence of seven NET proteins (designated NET-1 through NET-7), and studied expression in a panel of cell lines.

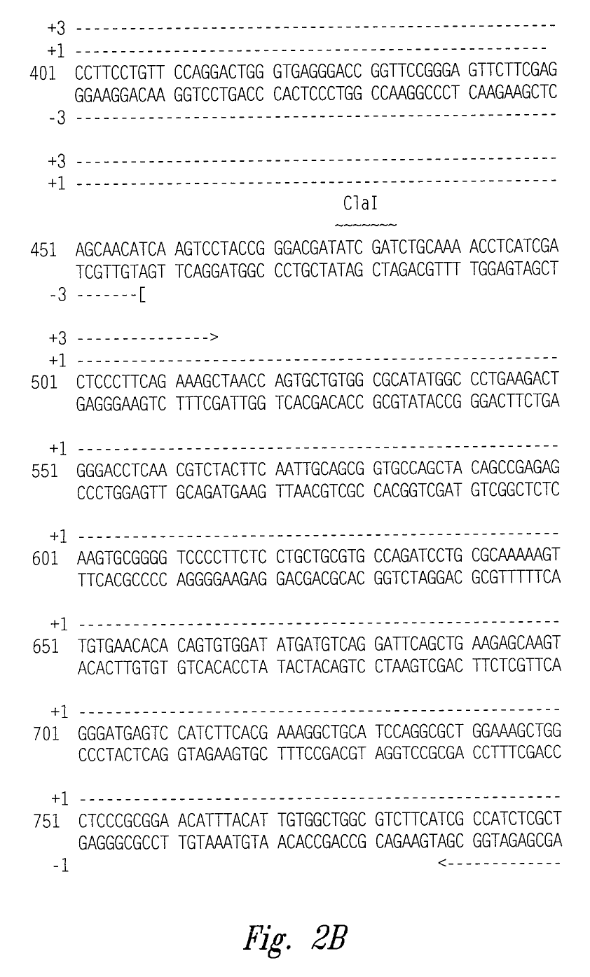

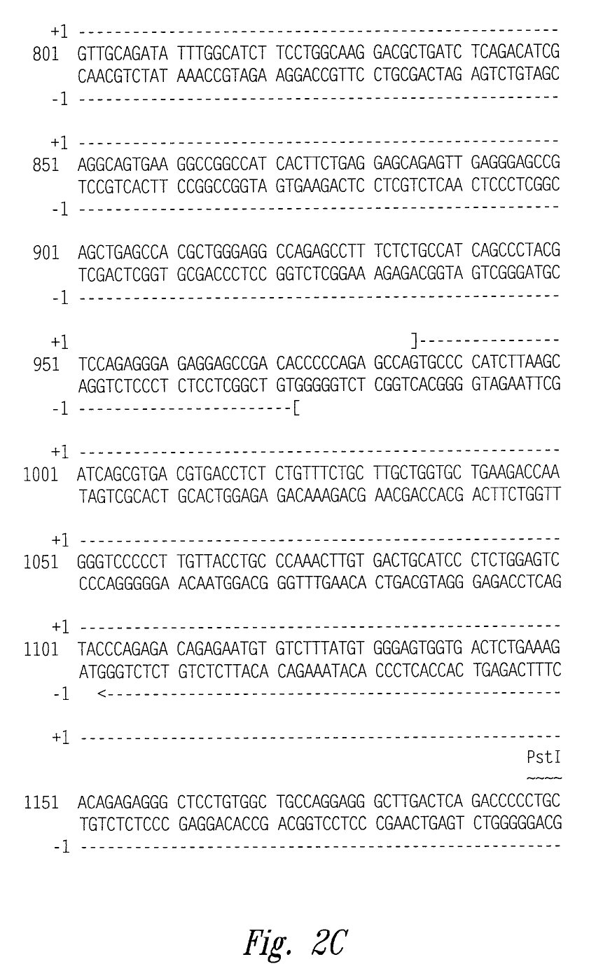

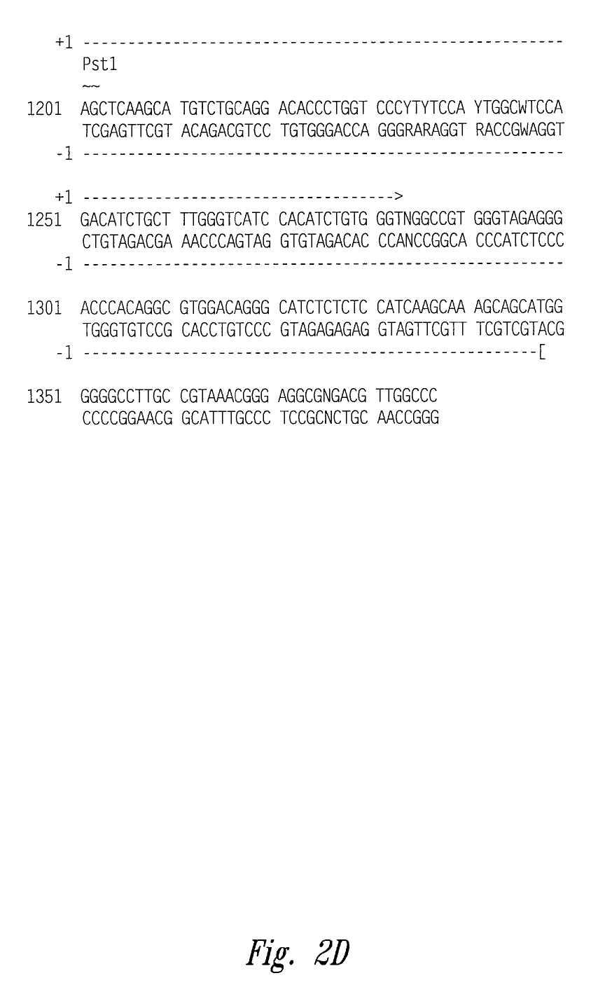

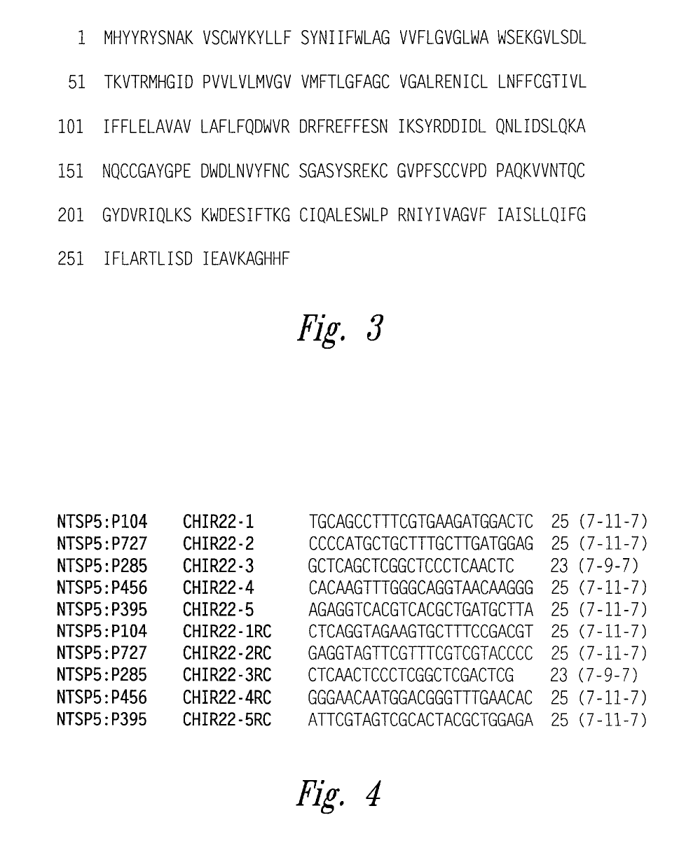

The present invention provides a new member of the tetraspan family, referred to herein as TSPAN-7. The TSPAN-7 of the invention was expressed in T lymphoid cell lines, but not by most B lymphoid cell lines studied. The cDNA is most homologous to NET-4, also known as TSPAN-5. The full-length CDNA sequence, SEQ ID NO:1, is provided in FIG. 2, and the encoded amino acid sequence, SEQ ID NO:2, is provided in FIG. 3. The invention further adds to the knowledge about the tetraspan family by disclosing that TSPAN-7 mRNA is differentially expressed in prostate cancer cell lines. Specifically, TSPAN-7 expression was over 9-fold higher in prostate cancer cell line WOca than in normal prostate cell line GRRpz. The GRRpz cell line refers to low passage (3 passages or fewer) human prostate cells. The WOca cell line refers to low passage (3 passages or fewer) human prostate cancer cells. TSPAN-7 therefore is a candidate for modulating growth, proliferation, migration, and metastasis of prostate cancer, hyperproliferative disorders, and other cancers. In diagnostic uses, the presence of prostate cancer cells can be detected using agents that bind to TSPAN-7 or an extracellular region thereof, such as antibodies.

To modulate TSPAN-7 expression, SW620 colon cancer cells were transfected with antisense oligonucleotides designed to specifically hybridize with TSPAN-7 polynucleotides. The oligonucleotides used herein are shown in SEQ ID NO:3-7 and are designated 22-1, 22-2, 22-3, 22-4, and 22-5, respectively. As a control, cells were transfected with corresponding reverse complement oligonucleotides designated 22-1RC, 22-2RC, 22-3RC, 22-4RC and 22-5RC (SEQ ID NO:8-12, respectively). mRNA levels in treated cells were analyzed and normalized to actin. As shown in FIG. 4, cell populations treated with four of the five antisense oligonucleotides (22-1, 22-2, 22-4 and 22-5) showed reduced mRNA levels relative to the levels in the corresponding RC-treated cells.

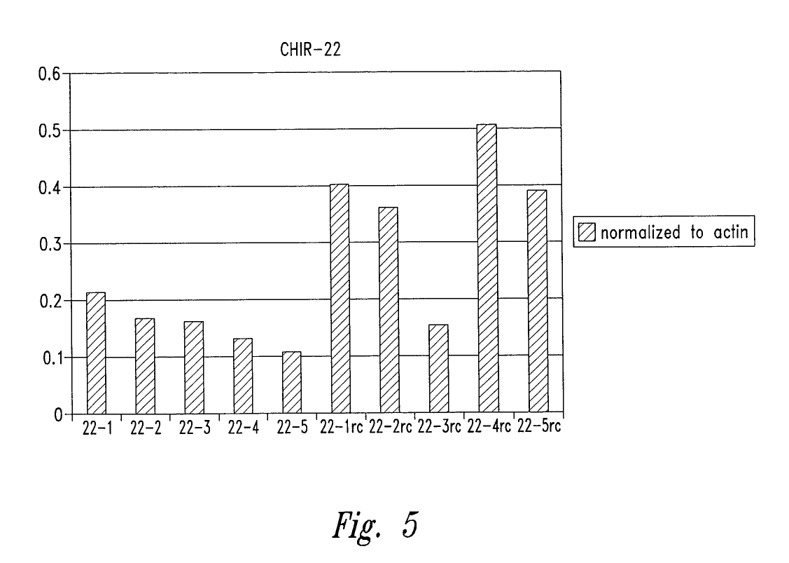

The greatest mRNA reduction was found in cells treated with 22-4 antisense, and this oligonucleotide was selected to measure the effect on SW620 cell proliferation. As shown in FIG. 5, untreated SW620 cells (WT) had an increase in fluorescence, indicative of total DNA levels and proliferation, from 1200 to 4250 between day 0 and day 4. Cells treated with 22-4RC also showed a steady rate of proliferation, from 1300 at day 0 to 3000 at day 4. In contrast, 22-4 antisense-treated cells remained at about 1000 from day 0 to day 2, to about 1300 at day 3 and to about 2300 at day 4. Thus, antisense inhibition of proliferation of SW620 cells correlated with decreased TSPAN-7 mRNA levels in the cells.

The NET protein superfamily was discovered in 1990, and as of 1997, 20 members had been identified. It has been suggested that one of the molecule's functions is to group specific cell-surface proteins, thereby increasing the formation and stability of functional signaling complexes. Maecker, H.T. et al., FASEB J. 11:428-442, 1997. FIG. 1 illustrates the schematic structure of tetraspan proteins. The information published to date indicates that some tetraspan proteins may play an inhibitory role in cancer development or growth, while other tetraspan proteins are expressed at a higher level in cancer cells. For example, expression of CD9 suppresses motility and metastasis in carcinoma cells (Ikeyama, S. et al., J. Exp. Med. 177:1231-1237, 1993), and CD9 expression is inversely correlated with metastasis in melanoma cells (Si, Z. et al., Int. J. Cancer 54:37-43, 1993). Reduction of CD9 expression correlates with poor prognosis in breast carcinoma (Miyake, M. et al., Cancer Res. 56:1244-1249, 1996). Expression of CD82 may suppress metastasis in prostate cancer cell lines (Dong, J. et al., Science 268:884-886, 1995).

Antisense oligonucleotides based on the polynucleotide sequence of TSPAN-7 therefore are specific inhibitors of TSPAN-7 expression, and this correlates with decreased proliferation of colon cancer cells. Antisense oligonucleotides are suitable for in vivo treatment of prostate cancer and other cancers in which increased TSPAN-7 expression plays a role in cell growth, migration, metastasis, and survival. However, the invention is not limited to use of antisense inhibitors. Based on the results herein, other compositions and methods for inhibiting TSPAN-7 expression or for modulating or inhibiting TSPAN-7 function are also suitable for regulating cell proliferation. Because TSPAN-7 is a transmembrane protein, antibodies are particularly suitable for inhibiting its effect.

TSPAN-7 has at least two extracellular domains. The first domain has the amino acid sequence:

| | |
|---|---|
| AWSEKGVLSDLTKVTRMHGIDPVV | (SEQ ID NO:13) |

The second domain has the amino acid sequence:

| FLFQDWVRDRFREFFESNIKSYRDDIDLQNLIDSLQKANQ | (SEQ ID |
|---|---|
| 20 60 | NO:14) |
| CCGAYGPEDWDLNVYFNCSGASYSREKCGVPFSCCVPDPA | |
| 80 100 | |
| QKVVNTQCGYDVRIQLKSKWDESIFTKGCIQALESWLPRN | |
| 120 140 | |

These domains are suitable for targeting therapeutic agents to cancer cells, such as prostate cancer cells, through the use of binding partners such as antibodies capable of specifically binding to SEQ ID NO:13 or 14, or to fragments thereof.

The present invention is directed generally to modulating TSPAN-7 expression and function in hyperproliferative disorders, such as in cancer cells, particularly in prostate cancer cells. More specifically, the invention disclosed herein provides inhibitors of TSPAN-7, including antisense polynucleotides and ribozymes, proteins or polypeptides, antibodies or fragments thereof and small molecules; compositions comprising TSPAN-7 inhibitors; methods of supplementing the chemotherapeutic and/or radiation effects on a mammalian cell, as well as methods of treating hyperproliferative disorders and neoplastic diseases. These methods have in common the administration to a mammalian cell of one or more TSPAN-7 inhibitor.

TSPAN-7 Polypeptides, Polynucleotides and Variants Thereof

Polypeptide Fragments

The invention provides polypeptide fragments of TSPAN-7. Polypeptide fragments of the invention can comprise at least 8, 10, 12, 15, 18, 19, 20, 25, 50, 75, 100, 125, 130, 150, 170, 180, 200, 225, 250, 260, 265, 267, and 269 contiguous amino acids selected from SEQ ID NO:2. Preferred fragments include SEQ ID NO:13, SEQ ID NO:14, and fragments thereof.

Biologically Active Variants

Variants of the protein and polypeptides disclosed herein can also occur. Variants can be naturally or non-naturally occurring. Naturally occurring variants are found in humans or other species and comprise amino acid sequences that are substantially identical to the amino acid sequence shown in SEQ ID NO:2. Prefered fragments include SEQ ID NO:13, SEQ ID NO:14, and fragments thereof. Species homologs of the protein can be obtained using subgenomic polynucleotides of the invention to make suitable probes or primers to screen cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants, specifically the tetraspan configuration (FIG. 1) and the interaction with other cell surface proteins, are also included here. Preferably, naturally or non-naturally occurring variants have amino acid sequences which are at least 85%, 90%, or 95% identical to the amino acid sequence shown in SEQ ID NO:2. More preferably, the molecules are at least 96%, 97%, 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in TSPAN-7 protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tytosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant.

Variants of the TSPAN-7 protein disclosed herein include glycosylated forms, aggregative conjugates with other molecules, and covalent conjugates with unrelated chemical moieties. Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

Preferably, amino acid changes in the TSPAN-7 protein or polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. Guidance for preparing variants can be found in FIG. 1 which indicates the location of conserved amino acids of the tetraspan family.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting protein or polypeptide variant. Properties and functions of TSPAN-7 protein or polypeptide variants are of the same type as a protein comprising the amino acid sequence encoded by the nucleotide sequence shown in SEQ ID NO:1, although the properties and functions of variants can differ in degree.

TSPAN-7 protein variants include glycosylated forms, aggregative conjugates with other molecules, and covalent conjugates with unrelated chemical moieties. TSPAN-7 protein variants also include allelic variants, species variants, and muteins. Truncations or deletions of regions which do not affect the differential expression or transmembrane configuration of the TSPAN-7 protein are also variants. Covalent variants can be prepared by linking functionalities to groups which are found in the amino acid chain or at the N- or C-terminal residue, as is known in the art.

It will be recognized in the art that some amino acid sequence of the TSPAN-7 protein of the invention can be varied without significant effect on the structure or function of the protein. If such differences in sequence are contemplated, it should be remembered that there are critical areas on the protein which determine activity (FIG. 1). In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the TSPAN-7 polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the TSPAN-7 of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Fusion Proteins

Fusion proteins comprising proteins or polypeptide fragments of TSPAN-7 can also be constructed. Fusion proteins are useful for generating antibodies against amino acid sequences and for use in various assay systems. For example, fusion proteins can be used to identify proteins which interact with TSPAN-7 or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of TSPAN-7 or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize the amino acid sequence shown in SEQ ID NO:2 or can be prepared from biologically active variants of SEQ ID NO:2, such as those described above. The first protein segment can consist of a full-length TSPAN-7.

Other first protein segments can consist of at least 8, 10, 12, 15, 18, 19, 20, 25, 50, 75, 100, 125, 130, 140, 160, 180, 200, 220, 240, 260, 265 or 269 contiguous amino acids selected from SEQ ID NO:2.

In specific embodiments, the first protein segment can be one or both of SEQ ID NO:13 and SEQ ID NO:14, or portions thereof. Preferred embodiments include amino acids 1-24, 2-25, 2-24, 3-25, or 3-24 of SEQ ID NO:13; or fragments of SEQ ID NO:13 of 8, 9, 10, 11, 12, 13, 14, 15, 16, 17, 18, 19, 20, or 21 contiguous amino acids. Other preferred embodiments include amino acids 1-119, 2-220, 2-119, 3-220, 3-119, or 4-220 of SEQ ID NO:14, or fragments of SEQ ID NO:14 having 10, 11, 12, 13, 14, 15, 20, 25, 30, 35, 40, 45, 50, 55, 60, 65, 70, 75, 80, 85, 90, 95, 100, 105, 110, or 115 contiguous amino acids.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence of SEQ ID NO:1 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Isolation and Production of TSPAN-7

TSPAN-7 is expressed in prostate cancer line WOca and can be extracted from this cell line or from other human cells, such as recombinant cells comprising SEQ ID NO:1, using standard biochemical methods. These methods include, but are not limited to, size exclusion chromatography, ammonium sulfate fractionation, ion exchange chromatography, affinity chromatography, crystallization, electrofocusing, and preparative gel electrophoresis. The isolated and purified protein or polypeptide is separated from other compounds which normally associate with the protein or polypeptide in a cell, such as other proteins, carbohydrates, lipids, or subcellular organelles. A preparation of isolated and purified protein or polypeptide is at least 80% pure; preferably, the preparations are 90%, 95%, or 99% pure. Purity of the preparations can be assessed by any means known in the art. For example, the purity of a preparation can be assessed by examining electrophoretograms of protein or polypeptide preparations at several pH values and at several polyacrylamide concentrations, as is known in the art.

Proteins, fusion proteins, or polypeptides of the invention can be produced by recombinant DNA methods. For production of recombinant proteins, fusion proteins, or polypeptides, a coding sequence of the nucleotide sequence shown in SEQ ID NO:1 can be expressed in prokaryotic or eukaryotic host cells using expression systems known in the art. These expression systems include bacterial, yeast, insect, and mammalian cells. The resulting expressed TSPAN-7 protein can then be purified from the culture medium or from extracts of the cultured cells using purification procedures known in the art.

It may be necessary to modify a protein produced in yeast or bacteria, for example by phosphorylation or glycosylation of the appropriate sites, in order to obtain a functional protein. Such covalent attachments can be made using known chemical or enzymatic methods.

TSPAN-7 protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize TSPAN-7. General means for the production of peptides, analogs or derivatives are outlined in Chemistry and Biochemistry of Amino Acids, Peptides, and Proteins—A Survey of Recent Developments (ed., Weinstein, B. 1983). Substitution of D-amino acids for the normal L-stereoisomer can be carried out to increase the half-life of the molecule. Variants can be similarly produced.

Polynucleotide Sequences

A gene that encodes the TSPAN-7 protein of the invention has the coding sequence shown in SEQ ID NO:1. Polynucleotide molecules of the invention contain less than a whole chromosome and can be single- or double-stranded. Preferably, the polynucleotide molecules are intron-free. Polynucleotide molecules of the invention can comprise at least 11, 15, 16, 18, 21, 25, 26, 30, 33, 42, 54, 60, 66, 72, 84, 90, 10, 120, 140, 160, 180, 200, 240, 300, 330, 400, 420, 500, 540, 600, 660, 700, 750, 800, 850, 900, 950, 1000, 1050, 1100, 1150, 1200, 1250, 1300, 1325, 1350 or 1374 or more contiguous nucleotides from SEQ ID NO:1, or the complements thereof. The complement of the nucleotide sequence shown in SEQ ID NO:1 is a contiguous nucleotide sequence that forms Watson-Crick base pairs with a contiguous nucleotide sequence as shown in SEQ ID NO:1.

Degenerate polynucleotide sequences which encode amino acid sequences of the TSPAN-7 protein and variants, as well as homologous nucleotide sequences which are at least 65%, 75%, 85%, 90%, 95%, 96%, 97%, 98%, or 99% identical to the nucleotide sequence shown in SEQ ID NO:1, are also polynucleotide molecules of the invention. Percent sequence identity is determined by any method known in the art, for example, using computer programs which employ the Smith-Waterman algorithm, such as the MPSRCH program (Oxford Molecular), using an affine gap search with the following parameters: a gap open penalty of 12 and a gap extension penalty of 1.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% base pair mismatches. More preferably, homologous nucleic acid strands contain 15-25% base pair mismatches, even more preferably 5-15% base pair mismatches.

The invention also provides polynucleotide probes that can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides from SEQ ID NO:1. Polynucleotide probes of the invention can comprise a detectable label, such as a radioisotopic, fluorescent, enzymatic, or chemiluminescent label.

Isolated genes corresponding to SEQ ID NO:1 are also provided. Standard molecular biology methods can be used to isolate a corresponding gene using the cDNA sequence provided herein. These methods include preparation of probes or primers from the nucleotide sequence shown in SEQ ID NO:1 for use in identifying or amplifying the genes from human genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

Polynucleotide Constructs

Polynucleotide molecules encoding TSPAN-7 protein or polypeptides can be used in a polynucleotide construct, such as a DNA or RNA construct. Polynucleotide molecules of the invention can be used, for example, in an expression construct to express all or a portion of a TSPAN-7 protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment that encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Host Cells

An expression construct can be introduced into a host cell. The host cell comprising the expression construct can be any suitable prokaryotic or eukaryotic cell. Expression systems in bacteria include those described in Chang et al., Nature (1978) 275: 615; Goeddel et al., Nature 281: 544 (1979); Goeddel et al., Nucleic Acids Res. 8: 4057 (1980); EP 36,776; U.S. Pat. No. 4,551,433; deBoer et al., Proc. Natl. Acad. Sci. USA 80: 21-25 (1983); and Siebenlist et al., Cell 20:269 (1980).

Expression systems in yeast include those described in Hinnen et al., Proc. Natl. Acad. Sci. USA 75:1929 (1978); Ito et al., J. Bacteriol. 153:163 (1983); Kurtz et al., Mol. Cell Biol. 6:142 (1986); Kunze et al., J Basic Microbiol. 25:141 (1985); Gleeson et al., J. Gen. Microbiol. 132:3459 (1986), Roggenkamp et al., Mol. Gen. Genet. 202:302 (1986); Das et al., J Bacteriol. 158.1165 (1984); De Louvencourt et al., J. Bacteriol. 154:737 (1983), Van den Berg et al., Bio/Technology 8:135 (1990); Kunze et al., J. Basic Microbiol. 25:141 (1985); Cregg et al., Mol. Cell. Biol. 5:3376 (1985); U.S. Pat. No. 4,837,148; U.S. Pat. No. 4,929,555; Beach and Nurse, Nature 300:706 (1981); Davidow et al., Curr Genet. 1p: 380 (1985); Gaillardin et al., Curr Genet. 10:49 (1985); Ballance et al., Biochem. Biophys. Res. Commun. 112:284-289 (1983); Tilburn et al., Gene 26:205-22 (1983); Yelton et al., Proc. Natl. Acad. Sci. USA 81:1470-1474 (1984); Kelly and Hynes, EMBO J. 4:475479 (1985); EP 244,234; and WO 91/00357.

Expression of heterologous genes in insects can be accomplished as described in U.S. Pat. No. 4,745,051; Friesen et al. “The Regulation of Baculovirus Gene Expression” in: The Molecular Biology Of Baculoviruses (ed., Doerfler, W 1986); EP 127,839; EP 155,476; Vlak et al., J. Gen. Virol. 69:765-776 (1988); Miller et al., Ann. Rev. Microbiol. 42:177 (1988); Carbonell et al., Gene 73: 409 (1988); Maeda et al., Nature 315:592-594 (1985); Lebacq-Verheyden et al., Mol. Cell Biol. 8:3129 (1988); Smith et al., Proc. Natl. Acad. Sci. USA 82:8404 (1985); Miyajima et al., Gene 58:273 (1987); and Martin et al., DNA 7:99 (1988). Numerous baculoviral strains and variants and corresponding permissive insect host cells from hosts are described in Luckow et al., Bio/Technology 6:47-55 (1988), Miller et al., in Genetic Engineering (ed., Setlow, J. K. et al.), Vol. 8 (Plenum Publishing, 1986), pp. 277-279; and Maeda et al., Nature 315:592-594 (1985).

Mammalian expression can be accomplished as described in Dijkema et al., EMBO J. 4:761 (1985); Gormanetal., Proc. Natl. Acad. Sci. USA 79:6777 (1982b); Boshart el al., Cell 41:521 (1985); and U.S. Pat. No. 4,399,216. Other features of mammalian expression can be facilitated as described in Ham and Wallace, Meth Enz. 58:44 (1979); Barnes and Sato, Anal. Biochem. 102:255 (1980); U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a TSPAN-7 can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides from the nucleotide sequence shown in SEQ ID NO:1. The transcription unit is located upstream to a coding sequence of the endogenous gene. The exogenous regulatory sequence directs transcription of the coding sequence of the endogenous gene.

Inhibitors of TSPAN-7 are Effective in Reducing TSPAN-7 Gene Expression

The present invention provides inhibitors of TSPAN-7. Inventive inhibitors include antisense molecules and ribozymes, proteins or polypeptides, antibodies or fragments thereof as well as small molecules. Each of these TSPAN-7 inhibitors shares the common feature that they reduce the expression and/or biological activity of TSPAN-7 and, as a consequence, inhibit cancer cell proliferation. In addition to the exemplary TSPAN-7 inhibitors disclosed herein, alternative inhibitors may be obtained through routine experimentation using methods specifically disclosed herein or otherwise readily available to and within the expertise of the skilled artisan.

Antisense Molecules and Ribozymes

As discussed above, TSPAN-7 inhibitors of the present invention include antisense molecules that, when administered to mammalian cells, are effective in reducing TSPAN-7 mRNA levels. Antisense molecules bind in a sequence-specific manner to nucleic acids, such as mRNA or DNA. When bound to mRNA that has complementary sequences, antisense molecules prevent translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith, and Clusel et al. Nucl. Acids Res. 21:3405-3411 (1993), which describes dumbbell antisense oligonucleotides).

Antisense technology can be used to control gene expression through triple-helix formation, which promotes the ability of the double helix to open sufficiently for the binding of polymerases, transcription factors or regulatory molecules. See Gee et al., In Huber and Carr, “Molecular and Immunologic Approaches,” Futura Publishing Co. (Mt. Kisco, N.Y.; 1994). Alternatively, an antisense molecule may be designed to hybridize with a control region of the TSPAN-7 gene, e.g., promoter, enhancer or transcription initiation site, and block transcription of the gene, or block translation by inhibiting binding of a transcript to ribosomes. See generally, Hirashima et al. in Molecular Biology of RNA: New Perspectives (M. Inouye and B. S. Dudock, eds., 1987 Academic Press, San Diego, p. 401); Oligonucleotides: Antisense Inhibitors of Gene Expression (J. S. Cohen, ed., 1989 MacMillan Press, London); Stein and Cheng, Science 261:1004-1012 (1993); WO 95/10607; U.S. Pat. No. 5,359,051; WO 92/06693; and EP-A2-612844, each of which is incorporated herein by reference.

Briefly, such molecules are constructed such that they are complementary to, and able to form Watson-Crick base pairs with, a region of transcribed TSPAN-7 mRNA sequence. The resultant double-stranded nucleic acid interferes with subsequent processing of the mRNA, thereby preventing protein synthesis. Antisense molecules according to the invention are composed of regions of contiguous nucleotides capable of hybridizing to SEQ ID NO:1 or the complement thereof. Preferred antisense molecules consist of 10, 15, 16, 17, 18, 19, 20, 21, 22, 23, 24, 25 or 26 contiguous nucleotides of SEQ ID NO:1, or the complement thereof.

In general, a portion of a sequence complementary to the TSPAN-7 coding region may be used to modulate gene expression. The nucleic acid sequence of the human TSPAN-7 cDNA is presented herein as SEQ ID NO:1. Alternatively, cDNA constructs that can be transcribed into antisense RNA may be introduced into cells or tissues to facilitate the production of antisense RNA. Thus, as used herein, the phrase “antisense molecules” broadly encompasses antisense oligonucleotides whether synthesized as DNA or RNA molecules as well as all plasmid constructs that, when introduced into a mammalian cell, promote the production of antisense RNA molecules. An antisense molecule may be used, as described herein, to inhibit expression of TSPAN-7 gene as well as any other gene that requires TSPAN-7 for its expression.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361; 5,625,050 and 5,958,773.

The antisense compounds of the invention can include modified bases as disclosed in U.S. Pat. No. 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5.491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in Example 1. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.) According to the present invention, cultures of SW620 cells were transfected with five different antisense oligonucleotides designed to target TSPAN-7. These oligonucleotides are shown in SEQ ID NO:3-7. The levels of mRNA corresponding to TSPAN-7 were measured in treated and control cells. SEQ ID NO:3, 4, 5 and 7 caused dramatic decreases in TSPAN-7 mRNA when normalized to actin mRNA levels.

Antisense molecules for use as described herein can be synthesized bv any method known to those of skill in this art including chemical synthesis by, for example, solid phase phosphoramidite chemical synthesis. See, e.g., WO 93/01286; U.S. Pat. No. 6,043,090; U.S. Pat. No. 5,218,088; U.S. Pat. No. 5,175,269; and U.S. Pat. No. 5,109,124, each of which is incorporated herein by reference. Alternatively, RNA molecules may be generated by in vitro or in vivo transcription of DNA sequences encoding the TSPAN-7 cDNA, or a portion thereof, provided that the DNA is incorporated into a vector downstream of a suitable RNA polymerase promoter (such as, e.g., T3, T7 or SP6). Large amounts of antisense RNA may be produced by incubating labeled nucleotides with a linearized TSPAN-7 cDNA fragment downstream of such a promoter in the presence of the appropriate RNA polymerase. Within certain embodiments, an antisense molecule of the present invention will comprise a sequence that is unique to the TSPAN-7 cDNA sequence of SEQ ID NO:1 or that can hybridize to the cDNA of SEQ ID NO:1 under conditions of high stringency. Within the context of the present invention, high stringency means standard hybridization conditions such as, e.g., 5XSSPE, 0.5% SDS at 65° C. or the equivalent thereof See Sambrook et al., supra and Molecular Biotechnology: Principles and Applications of Recombinant DNA, supra, incorporated herein by reference.

Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate. phosphate esters, and other such linkages (see, e.g., Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. 17(12):4863-4871 (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein, in: Oligodeoxynucleotides. Antisense Inhibitors of Gene Expression, Cohen, Ed, Macmillan Press, London, pp. 97-117 (1989); Jager et al., Biochemistry 27:7237-7246 (1988)). Possible additional or alternative modifications include, but are not limited to, the addition of flanking sequences at the 5′ and/or 3′ ends and/or the inclusion of nontraditional bases such as inosine, queosine and wybutosine, as well as acetyl- methyl-, thio- and other modified forms of adenine, cytidine, guanine, thymine and uridine.

Exemplary antisense molecules of the invention include:

the 20-mer polynucleotides having nucleotides 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142,124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228,210-229,211-230,212-231, 213-232, 214-233,215-234, 216-235,217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305,287-306,288-307, 289-308,290-309, 291-310,292-311, 293-312,294-313, 295-314, 296-315,297-316, 298-317,299-318, 300-319, 301-320, 302-321,303-322, 304-323, 305-324, 306-325, 307-326, 308-327, 309-328, 310-329, 311-330, 312-331, 313-332, 314-333, 315-334,316-335, 317-336, 318-337, 319-338, 320-339, 321-340, 322-341,323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412,394-413, 395-414,396-415, 397-416, 398-417, 399-418,400-419, 401-420, 402-421, 403-422, 404-423, 405-424, 406-425, 407-426, 408-427, 409-428, 410-429, 411-430, 412-431, 413-432,414-433, 415-434,416-435, 417-436,418-437,419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609,591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724,706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810,792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403,1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, or 1400-1419 of SEQ ID NO:1 or the complement thereof,

and the 25-mer polynucleotides having nucleotides: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294,271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405,382-406, 383-407, 384-408, 385-409, 386-410, 387-411,388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432,409-433,410-434, 411-435,412-436, 413-437,414-438, 415-439,416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712,689-713, 690-714,691-715, 692-716, 693-717, 694-718, 695-719, 696-720, 697-721, 698-722, 699-723, 700-724, 701-725,702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807,784-808, 785-809, 786-810,787-811, 788-812, 789-813, 790-814, 791-815, 792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931,908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018,995-1019, 996-1020,997-1021, 998-1022,999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037,1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287,1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302,1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, or 1400-1424 of SEQ ID NO:1, or the complement thereof.

Within alternate embodiments of the present invention, TSPAN-7 inhibitors may be ribozymes. A ribozyme is an RNA molecule that specifically cleaves RNA substrates, such as mRNA, resulting in specific inhibition or interference with cellular gene expression. As used herein, the term “ribozymes” includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell 48:211-220 (1987); Haseloff and Gerlach, Nature 328:596-600 (1988); Walbot and Bruening, Nature 334:196 (1988); Haseloff and Gerlach, Nature 334:585 (1988)); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such TSPAN-7 mRNA-specific ribozyme, or a nucleic acid encoding such a ribozyme, may be delivered to a host cell to effect inhibition of TSPAN-7 gene expression. Ribozymes and the like may therefore be delivered to the host cells by DNA encoding the ribozyme linked to a eukaryotic promoter, such as a eukaryotic viral promoter, such that upon introduction into the nucleus, the ribozyme will be directly transcribed.

Proteins and Polvpeptides

In addition to the antisense molecules and ribozymes disclosed herein, TSPAN-7 inhibitors of the present invention also include proteins or polypeptides that are effective in either reducing TSPAN-7 gene expression or in decreasing one or more of TSPAN-7's biological activities. A variety of methods are readily available in the art by which the skilled artisan may, through routine experimentation, rapidly identify such TSPAN-7 inhibitors. The present invention is not limited by the following exemplary methodologies.

TSPAN-7 is believed to exert a biological effect by interacting with other cell surface proteins. Inhibitors of TSPAN-7's biological activities include those proteins and/or polypeptides that interfere with TSPAN-7's activity. Such interference may occur through direct interaction with TSPAN-7 or indirectly through non- or un-competitive inhibition such as via binding to an allosteric site. Accordingly, available methods for identifying proteins and/or polypeptides that bind to TSPAN-7 may be employed to identify lead compounds that may, through the methodology disclosed herein, be characterized for their TSPAN-7 inhibitory activity.

A vast body of literature is available to the skilled artisan that describes methods for detecting and analyzing protein-protein interactions. Reviewed in Phizicky, E. M. et al., Microbiological Reviews 59:94-123 (1995) incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive TSPAN-7 inhibitors may be identified through biological screening assays that rely on the direct interaction between the TSPAN-7 protein and a panel or library of potential inhibitor proteins. Biological screening methodologies, including the various “n-hybrid technologies,” are described in, for example, Vidal, M. et al., Nucl. Acids Res. 27(4):919-929 (1999); Frederickson, R. M., Curr Opin. Biotechnol. 9(1):90-6 (1998); Brachmann, R. K. et al., Curr Opin. Biotechnol. 8(5):561-568 (1997); and White, M. A., Proc. Natl. Acad. Sci. U.S.A. 93:10001-10003 (1996) each of which is incorporated herein by reference.

The two-hybrid screening methodology may be employed to search new or existing target cDNA libraries for TSPAN-7 binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems is available to the skilled artisan that most commonly employ either the yeast Gal4 or E. coil LexA DNA-binding domain (BD) and the yeast Gal4 or herpes simplex virus VP16 transcriptional activation domain. Chien, C. -T. et al., Proc. Natl. Acad. Sci. U.S.A. 88:9578-9582 (1991); Dalton, S. et al., Cell 68:597-612 (1992); Durfee, T. K. et al., Genes Dev. 7:555-569 (1993); Vojtek, A. B. et al., Cell 74:205-214 (1993); and Zervos, A. S. et al., Cell 72:223-232 (1993). Commonly used reporter genes include the E. coli lacZ gene as well as selectable yeast genes such as HIS3 and LEU2. Fields, S. et al., Nature (London) 340:245-246 (1989); Durfee, T. K., supra; and Zervos, A. S., supra. A wide variety of activation domain libraries are readily available in the art such that the screening for interacting proteins may be performed through routine experimentation.

Suitable bait proteins for the identification of TSPAN-7 interacting proteins may be designed based on the TSPAN-7 cDNA sequence (SEQ ID NO:1). Such bait proteins include either the full-length TSPAN-7 protein or fragments thereof. Particular regions include those encoding SEQ ID NO:13 and SEQ ID NO:14.

Plasmid vectors, such as, e.g., pBTM116 and pAS2-1, for preparing TSPAN-7 bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

TSPAN-7 inhibitors of the present invention may alternatively be identified through one of the physical or biochemical methods available in the art for detecting protein-protein interactions.

Through the protein affinity chromatography methodology, lead compounds to be tested as potential TSPAN-7 inhibitors may be identified by virtue of their specific retention to TSPAN-7 when either covalently or non-covalently coupled to a solid matrix such as, e.g., Sepharose beads. The preparation of protein affinity columns is described in, for example, Beeckmans, S. et al., Eur J. Biochem. 117:527-535 (1981) and Formosa, T. et al., Methods Enzymol. 208:24-45 (1991). Cell lysates containing the full complement of cellular proteins, or fractions enriched for cell membrane proteins that may interact with TSPAN-7, may be passed through the TSPAN-7 affinity column. Proteins having a high affinity for TSPAN-7 will be specifically retained under low-salt conditions while the majority of cellular proteins will pass through the column. Such high affinity proteins may be eluted from the immobilized TSPAN-7 under conditions of high-salt, with chaotropic solvents or with sodium dodecyl sulfate (SDS). In some embodiments, it may be preferred to radiolabel the cells prior to preparing the lysate as an aid in identifying the TSPAN-7 specific binding proteins. Methods for radiolabeling mammalian cells are well known in the art and are provided, e.g., in Sopta, M. et al., J. Biol. Chem. 260:10353-10360 (1985).

Suitable TSPAN-7 proteins for affinity chromatography may be fused to a protein or polypeptide to permit rapid purification on an appropriate affinity resin. For example, the TSPAN-7 cDNA may be fused to the coding region for glutathione S-transferase (GST) which facilitates the adsorption of fusion proteins to glutathione-agarose columns. Smith et al., Gene 67:31-40 (1988). Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of TSPAN-7 fusion proteins is the epitope for the influenza virus hemagglutinin (HA) against which monoclonal antibodies are readily available and from which antibodies an affinity column may be prepared.

Proteins that are specifically retained on a TSPAN-7 affinity column may be identified after subjecting to SDS polyacrylamide gel electrophoresis (SDS-PAGE). Thus, where cells are radiolabeled prior to the preparation of cell lysates and passage through the TSPAN-7 affinity column, proteins having high affinity for TSPAN-7 may be detected by autoradiography. The identity of TSPAN-7 specific binding proteins may be determined by protein sequencing techniques that are readily available to the skilled artisan, such as Matthews, C. K. et al., Biochemistry, The Benjamin/Cummings Publishing Company, Inc. pp. 166-170 (1990).

Production of Antagonists

The methods and compositions of the present invention use, or incorporate, a TSPAN-7 antagonist. Accordingly, methods for generating such antagonists are described here. The TSPAN-7 to be used for production of, or screening for, antagonist(s) may be, e.g., a soluble form of the protein or a portion thereof, containing the desired epitope, for example, SEQ ID NO:13 or SEQ ID NO:14. Alternatively, or additionally, cells expressing TSPAN-7 on their cell surface can be used to generate, or screen for, antagonist(s).

While preferred antagonists include antibodies and antisense molecules, as discussed below, antagonists other than antibodies and antisense molecules are contemplated herein. For example, the antagonist may comprise a small molecule antagonist optionally fused to, or conjugated with, a cytotoxic agent. Libraries of small molecules may be screened against TSPAN-7 or TSPAN-7 expressing cells in order to identify a small molecule which binds to that antigen. The small molecule may further be screened for its antagonistic properties and/or conjugated with a cytotoxic agent.

The antagonist may also be a peptide generated by rational design or by phage display (see, e.g., W098/35036 published Aug. 13, 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide. Methods of identifying peptides that can serve as antagonists to cell surface proteins are based on methods disclosed in, for example, U.S. Pat. Nos. 6,110,747; 6,203,788; and 6,248,864. Preferred peptide antagonists of TSPAN-7 will include peptides, peptide mimetics, and cyclic peptides. Additionally, the antagonist may be an antisense oligonucleotide or ribozyme. A description follows as to exemplary techniques for the production of the antibody antagonists used in accordance with the present invention.

TSPAN-7 inhibitors of the present invention include antibodies and/or antibody fragments that are effective in reducing TSPAN-7 gene expression and/or biological activity, such as by interfering with TSPAN-7 interaction with other cell membrane proteins. Suitable antibodies may be monoclonal, polyclonal or humanized monoclonal antibodies. Antibodies may be derived by conventional hybridoma based methodology; from antisera isolated from TSPAN-7 inoculated animals; or through recombinant DNA technology. Alternatively, inventive antibodies or antibody fragments may be identified in vitro by use of one or more of the readily available phage display libraries. Exemplary methods are disclosed herein.

The fragments of TSPAN-7 referred to herein are, for example, polypeptides having at least 8, 9, 10, 12, 15, or 20 contiguous amino acids of SEQ ID NO:2. Exemplary polypeptides includes the following 9-mer polypeptides of the 270 amino acid TSPAN-7:

amino acids 1-9, 2-10, 3-11, 4-12, 5-13, 6-14, 7-15, 8-16, 9-17, 10-18, 11-19, 12-20, 13-21, 14-22, 15-23, 16-24, 17-25, 18-26, 19-27, 20-28, 21-29, 22-30, 23-31, 24-32, 25-33, 26-34, 27-35, 28-36, 29-37, 30-38, 31-39, 32-40, 33-41, 34-42, 35-43, 36-44, 37-45, 38-46, 39-47, 40-48, 41-49, 42-50, 43-51, 44-52, 45-53, 46-54, 47-55, 48-56, 49-57, 50-58, 51-59, 52-60, 53-61, 54-62, 55-63, 56-64, 57-65, 58-66, 59-67, 60-68, 61-69, 62-70, 63-71, 64-72, 65-73, 66-74, 67-75, 68-76, 69-77, 70-78, 71-79, 72-80, 73-81, 74-82, 75-83, 76-84, 77-85, 78-86, 79-87, 80-88, 81-89, 82-90, 83-91, 84-92, 85-93, 86-94, 87-95, 88-96, 89-97, 90-98, 91-99, 92-100, 93-101, 94-102, 95-103, 96-104, 97-105, 98-106, 99-107, 100-108, 101-109, 102-110, 103-111, 104-112, 105-113, 106-114, 107-115, 108-116, 109-117, 110-118, 111-119, 112-120, 113-121, 114-122, 115-123, 116-124, 117-125, 118-126, 119-127, 120-128, 121-129, 122-130, 123-131, 124-132, 125-133, 126-134, 127-135, 128-136, 129-137, 130-138, 131-139, 132-140, 133-141, 134-142, 135-143, 136-144, 137-145, 138-146, 139-147, 140-148, 141-149, 142-150, 143-151, 144-152, 145-153, 146-154, 147-155, 148-156, 149-157, 150-158, 151-159, 152-160, 153-161, 154-162, 155-163, 156-164, 157-165, 158-166, 159-167, 160-168, 161-169, 162-170, 163-171, 164-172, 165-173, 166-174, 167-175, 168-176, 169-177, 170-178, 171-179, 172-180, 173-181, 174-182, 175-183, 176-184, 177-185, 178-186, 179-187, 180-188, 181-189, 182-190, 183-191, 184-192, 185-193, 186-194, 187-195, 188-196, 189-197, 190-198, 191-199, 192-200, 193-201, 194-202, 195-203, 196-204, 197-205, 198-206, 199-207, 200-208, 201-209, 202-210, 203-211, 204-212, 205-213, 206-214, 207-215, 208-216, 209-217, 210-218, 211-219, 212-220, 213-221, 214-222, 215-223, 216-224, 217-225, 218-226, 219-227, 220-228, 221-229, 222-230, 223-231, 224-232, 225-233, 226-234, 227-235, 228-236, 229-237, 230-238, 231-239, 232-240, 233-241, 234-242, 235-243, 236-244, 237-245, 238- 246, 239-247, 240-248, 241-249, 242-250, 243-251, 244-252, 245-253, 246-254, 247-255, 248-256, 249-257, 250-258, 251-259, 252-260, 253-261, 254-262, 255-263, 256-264, 257-265, 258-266, 259-267, 260-268, 261-269, and 262-270 of SEQ ID NO:2.

12-mer polypeptides of the 270 amino acid TSPAN-7 include: amino acids 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, 22-33, 23-34, 24-35, 25-36, 26-37, 27-38, 28-39, 29-40, 30-41, 31-42, 32-43, 33-44, 34-45, 35-46, 36-47, 37-48, 38-49, 39-50, 40-51, 41-52, 42-53, 43-54, 44-55, 45-56, 46-57, 47-58, 48-59, 49-60, 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248- 259, 249-260, 250-261, 251-262, 252-263, 253-264, 254-265, 255-266, 256-267, 257-268,258-269, and 259-270 of SEQ ID NO:2.

15-mer polypeptides of the 270 amino acid TSPAN-7 include: amino acids 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 46-60, 47-61, 48-62, 49-63, 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244 -258, 245-259, 246-260, 247-261, 248-262, 249-263, 250-264, 251-265, 252-266, 253-267, 254-268, 255-269, and 256-270 of SEQ ID NO:2.

20-mer polypeptides of the 270 amino acid TSPAN-7 include: amino acids 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, and 251-270 of SEQ ID NO:2.

Polyclonal antibodies

Polyclonal antibodies are preferably raised in animals by multiple subcutaneous (sc), intraperitoneal (ip) or intramuscular (im) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, malcimidobenzoyl sulfosuccinimide ester (conjugation through cysteine residues), N-hydroxysuccinimide (through lysine residues), glutaraldehyde, succinic anhydride, SOC12, or R1N—C—NR, where R and R1 are different alkyl groups. Animals are immunized against the antigen, immunogenic conjugates, or derivatives by combining, e.g., 100 pg or 5 wg of the protein or conjugate (for rabbits or mice, respectively) with 3 volumes of Freund's complete adjuvant and injecting the solution intradennally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies

Monoclonal antibodies are obtained from a population of substantially homogeneous antibodies, i. e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567),

In the hybridoma method, a mouse or other appropriate host animal, such as a rabbit or hamster, is immunized as described to elicit lymphocytes that produce or are capable of producing antibodies that will specifically bind to the protein used for immunization. Alternatively, lymphocytes tray be immunized in vitro. Lymphocytes then are fused with myeloma cells using a suitable fusing agent, such as polyethylene glycol, to form a hybridoma cell [Goding, Monoclonal Antibodies: Principles and Practice, pp. 59-103 (Academic Press, 1986)].

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors (eleven) available from the Salk Institute Cell Distribution Center, San Diego, Calif. USA, and SP-2 or X63-Ag8-653 cells available from the American Type Culture Collection, Manassas, Va., USA. Human myeloma and mouse human heteromyeloma cell lines also have been described for the production of human monoclonal antibodies [Kozbor, J. Immunol., 133:3001 (1984); Brodeur et al., Monoclonal Antibody Production Techniques and Applications, pp. 51-63 (Marcel Dekker, Inc., New York, 1987)].

Culture medium in which hybridoma cells are growing is assayed for the production of monoclonal antibodies having the requisite specificity, e.g., by an in vitro binding assay such as enzyme-linked immunoabsorbent assay (ELISA) or radioimmunoassay (RIA). The location of the cells that express the antibody may be detected by FACS. Thereafter, hybridoma clones may be subcloned by limiting dilution procedures and grown by standard methods (Goding, Monoclonal Antibodies: Principles and Practice, Academic Press (1986) pp. 59-103). Suitable culture media for this purpose include, for example, DMBM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of marine (antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr Opinion in Immunol., 5:256-262 (1993) and Phickthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol, 222:581-597 (1991) describe the isolation of marine and human antibodies, respectively, using phage libraries. Subsequent publications describe the production of high affinity (nM range) human antibodies by chain shuffling (Marks et al., Biotechnology, 10:779-783 (1992)), as well as combinatorial infection and in vivo recombination as a strategy for constructing very large phage libraries (Waterhouse et al., Nuc. Acids. Res., 21:2265-2266 (1993)). Thus, those techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous marine sequences (U.S. Pat. No. 4,816,567; Morrison, et al, Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted far the constant domains of an antibody, or they are substituted for the variable domains of one antigen-combining site of an antibody to create a chimeric bivalent antibody comprising one antigen-combining site having specificity for an antigen and another antigen combining site having specificity for a different antigen.

Additionally, recombinant antibodies against TSPAN-7 can be produced in transgenic animals, e.g., as described in various patents many of which are assigned to Abgenix and Medarex. For example, recombinant antibodies can be expressed in transgenic animals, e.g., rodents as disclosed in any of U.S. Pat. Nos. 5,877,397, 5,874,299, 5,814,318, 5,789,650, 5,770,429, 5,661,016, 5,633,425, 5,625,126, 5,569,825, 5,545,806, 6,162,963, 6,150,584, 6,130,364, 6,114,598, 6,091,001, 5,939,598. Alternatively, recombinant antibodies can be expressed in the milk of transgenic animals as discussed in U.S. Pat. No. 5,849,992 or U.S. Pat. No. 5,827,690 which are assigned to Pfarmin, incorporated by reference herein.

Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. Nature, 321:522-525 (1986); Riechmann et al, Nature, 332:323-327 (1988); Verhoeyen et al, Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol, 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol, 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences.

Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

As an alternative to humanization, human antibodies can be generated. As discussed above, the production of antibodies, particularly human antibodies in transgenic animals is known. For a ample, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Mad Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology, 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mal. Biol., 222:581-597 (1991), or Griffith et al., EMBO J., 12:725-734 (1993). See also, U.S. Pat. Nos. 5,565,332 and 5,573,905. Human antibodies may also be generated by in vitro activated B cells (see U.S. Pat. Nos. 5,567,610 and 5,229,275).

Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods, 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab-SH Fragments can be directly recovered from E. coli and chemically coupled to form F(ab′)₂ fragments [Carter et al., Bio/Technology, 10:163-167 (1992)]. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody,” e.g., as described in U.S. Pat. No. 5,641,870 for example. Such linear antibody fragments may be monospecific or bispecific.

Bispecific Antibodies

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CHI) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Recent progress has facilitated the direct recovery of Fab′-SH fragments from E. coli, which can be chemically coupled to form bispecific antibodies. Shalaby et al. J. Exp. Med,, 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbH2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispectfic antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain.

Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Antibodies with more than two valencies are contemplated. For example, trispecific antibodies can be prepared. Tutt et al., J. Immunol., 147: 60 (1991).

Phage display libraries for the production of high-affinity antibodies are described in, for example, Hoogenboom, H. R. et al., Immunotechnology 4(1):1-20 (1998); Hoogenboom, H. R., Trends Biotechnol. 15:62-70 (1997) and McGuinness, B. et al., Nature Bio. Technol. 14:1149-1154 (1996) each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature 348:552-554 (1990) which is incorporated herein by reference. In short, the coding sequence of the antibody variable region is fused to the amino terminus of a phage minor coat protein (pill). Expression of the antibody variable region-pill fusion construct results in the antibody's “display” on the phage surface with the corresponding genetic material encompassed within the phage particle.

TSPAN-7 protein suitable for screening a phage library may be obtained by, for example, expression in baculovirus Sf9 cells as described, supra. Alternatively, the TSPAN-7 coding region may be PCR amplified using primers specific to the desired region of the TSPAN-7 protein. For example, where the inhibitor is directed against TSPAN-7's kinase domain, fragments may be amplified that encode the amino acid sequence flanking lysine 40 in the active site. As discussed above, the TSPAN-7 protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-TSPAN-7 binding properties may subsequently be isolated by successive panning, in the case of a solid matrix, or by affinity adsorption to a TSPAN-7 antigen column. Phage having the desired TSPAN-7 inhibitory activities may be reintroduced into bacteria by infection and propagated by standard methods known to those skilled in the art. See Hoogenboom, H. R., Trends Biotechnol., supra for a review of methods for screening for positive antibody-pIII phage.

Small Molecules

The present invention also provides small molecule TSPAN-7 inhibitors that may be readily identified through routine application of high-throughput screening (HTS) methodologies. Reviewed by Persidis, A., Nature Biotechnology 16:488-489 (1998). HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. See, e.g., Gonzalez, J. E. et al., Curr Opin. Biotechnol. 9(6):624-631 (1998) incorporated herein by reference.

Methods for Assessing the Efficacy of TSPAN-7 Inhibitors

Lead molecules or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to inhibit TSPAN-7 gene expression or biological activity. As discussed in further detail in the Examples provided below, TSPAN-7 inhibitors of the present invention are effective in reducing not only TSPAN-7 expression levels but also reducing SW620 cell proliferation. Thus, the present invention further discloses methods that permit the skilled artisan to assess the effect of candidate inhibitors on each of these parameters.

As noted above and as presented in the Examples, candidate TSPAN-7 inhibitors may be tested by administration to cells that either express endogenous TSPAN-7 or that are made to express TSPAN-7 by transfection of a mammalian cell with a recombinant TSPAN-7 plasmid construct.

Effective TSPAN-7 inhibitory molecules will be effective in reducing the levels of TSPAN-7 mRNA as determined, e.g., by Northern blot or RT-PCR analysis. For a general description of these procedures, see, e.g., Sambrook et al., Molecular Cloning: A Laboratory Manual Cold Spring Harbor Press (1989) and Molecular Biotechnology: Principles and Applications of Recombinant DNA, ASM Press (ed. Click, B. R. and Pasternak, J. J. 1998) incorporated herein by reference. The effectiveness of a given candidate antisense molecule may be assessed by comparison with a control “antisense” molecule known to have no substantial effect on TSPAN-7 expression when administered to a mammalian cell. Exemplary control molecules include the RC oligonucleotides disclosed in the Examples.

TSPAN-7 inhibitors effective in reducing TSPAN-7 gene expression or cell proliferation by one or more of the methods discussed above may be further characterized in vivo for efficacy in one of the readily available animal model systems. The various animal model systems for study of cancer and genetic instability associated genes are discussed in, for example, Donehower, L. A. Cancer Surveys 29:329-352 (1997), incorporated herein by reference.

Use of TSPAN-7 Inhibitors to Reduce the Severity of Cancer Therapy Side Effects

It has been discovered, as part of the present invention, that TSPAN-7 inhibitors are effective in reducing tumor cell growth. Accordingly, TSPAN-7 inhibitors may be effective as drugs for supplementing cancer therapy, such as radiation therapy or chemotherapy.

Lead compounds may be identified by the methods provided herein or by other suitable methods available in the art.

Administration of TSPAN-7 Inhibitors and Compositions Thereof

The present invention provides TSPAN-7 inhibitors and compositions comprising one or more TSPAN-7 inhibitor as well as methods that employ these inventive inhibitors in in vivo, ex vivo, and in vitro applications where it is advantageous to reduce or eliminate the expression or activity of TSPAN-7 or a functionally downstream molecule. As indicated above, TSPAN-7 inhibitor based compositions will find utility in the treatment of neoplastic disease and related conditions where treatment regimens are improved by radiation hypersensitivity of tumor cells. Alternatively, TSPAN-7 inhibitors may find use as drugs for reducing the side effects of, e.g., cancer therapeutics and other agents.

Compositions may be administered parenterally, topically, orally or locally for therapeutic treatment. Preferably, the compositions are administered orally or parenterally, i.e., intravenously, intraperitoneally, intradermally or intramuscularly.

Inventive compositions will include one or more TSPAN-7 inhibitor and may further comprise a pharmaceutically acceptable carrier or excipient. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

TSPAN-7 inhibitors useful in the treatment of disease in mammals will often be prepared substantially free of other naturally occurring immunoglobulins or other biological molecules. Preferred TSPAN-7 inhibitors will also exhibit minimal toxicity when administered to a mammal.

The compositions of the invention may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride and stabilizers (e.g., 1-20% maltose, etc.).

The selection of the appropriate method for administering TSPAN-7 inhibitors of the present invention will depend on the nature of the application envisioned as well as the nature of the TSPAN-7 inhibitor. Thus, for example, the precise methodology for administering a TSPAN-7 inhibitor will depend upon whether it is an antisense molecule, a protein and/or peptide, an antibody or antibody fragment or a small molecule. Other considerations include, for example, whether the TSPAN-7 inhibitor will be used to treat cancer cell proliferation or to supplement other cancer therapeutics.

A variety of methods are available in the art for the administration of antisense molecules. Exemplary methods include gene delivery techniques, including both viral and non-viral based methods as well as liposome mediated delivery methods.

By these methodologies, substantial therapeutic benefit may be achieved despite transfection efficiencies significantly less than 100%, transient retention of the transfected inhibitor and/or existence of a subpopulation of target cells refractory to therapy.

Gene delivery methodology may be used to directly knock-out endogenous TSPAN-7 within tumor cells thereby inhibiting cell proliferation. For example, the TSPAN-7 gene may be targeted by transfection of a gene delivery vector carrying a TSPAN-7 inhibitor. Preferential transfection into or expression within tumor cells may be achieved through use of a tissue-specific or cell cycle-specific promoter, such as, e.g., promoters for prostate-specific antigen or for immunoglobulin genes (Vile, R. G. et al., Cancer Res. 53:962-967 (1993) and Vile, R. G., Semin. Cancer Biol. 5:437-443 (1994)) or through the use of trophic viruses that are confined to particular organs or structures, such as, e.g., a replication selective and neurotrophic virus that can only infect proliferating cells in the central nervous system.

Thus, to achieve therapeutic benefit, TSPAN-7 within the tumor cells should be preferentially inhibited. This can be accomplished by transfecting a gene expressing a TSPAN-7 inhibitor, a TSPAN-7 antisense molecule, a TSPAN-7 gene specific repressor, or an inhibitor of the protein product of the TSPAN-7 gene.

As used herein, the phrase “gene delivery vector” refers generally to a nucleic acid construct that carries and, within certain embodiments, is capable of directing the expression of an antisense molecule of interest, as described in, for example, Molecular Biotechnology: Principles and Applications of Recombinant DNA, Ch. 21, pp. 555-590 (ed. B. P. Glick and J. J. Pasternak, 2nd ed. 1998); Jolly, Cancer Gene Ther 1:51-64 (1994); Kimura, Human Gene Ther 5:845-852 (1994); Connelly, Human Gene Ther 6:185-193 (1995); and Kaplitt, Nat. Gen 6:148-153 (1994).

A number of virus and non-virus based gene delivery vector systems have been described that are suitable for the administration of TSPAN-7 inhibitors. Virus based gene delivery systems include, but are not limited to retrovirus, such as Moloney murine leukemia virus, spumaviruses and lentiviruses; adenovirus; adeno-associated virus; and herpes-simplex virus vector systems. Viruses of each type are readily available from depositories or collections such as the American Type Culture Collection (ATCC; 10801 University Boulevard, Manassas, Va. 20110-2209) or may be isolated from known sources using commonly available materials and techniques.

The gene delivery vector systems of the present invention will find applications both in in vivo as well as ex vivo therapeutic regimens. Each of these applications is described in further detail below.

1. Retroviral Gene Delivery Vector Systems

Within one aspect of the present invention, retroviral gene delivery vectors are provided that are constructed to carry or express a TSPAN-7 inhibitory antisense molecule. As used herein, the term “TSPAN-7 inhibitory antisense molecule” refers generally to a nucleic acid sequence having TSPAN-7 inhibitory activity. More specifically, such antisense molecules will reduce TSPAN-7 gene expression and will inhibit target cell proliferation. Retroviral gene delivery vectors of the present invention may be readily constructed from a wide variety of retroviruses, including for example, B, C, and D type retroviruses as well as spumaviruses and lentiviruses. See RNA Tumor Viruses, Cold Spring Harbor Laboratory (2nd ed. 1985).

Any of the above retroviruses may be readily utilized in order to assemble or construct retroviral gene delivery vectors given the disclosure provided herein, and standard recombinant DNA techniques. See, e.g. Sambrook et al, Molecular Cloning. A Laboratory Manual, Cold Spring Harbor Laboratory Press (2d ed. 1989) and Kunkle, Proc. Natl. Acad. Sci. USA. 82:488 (1985). In addition, within certain embodiments of the invention, portions of the retroviral gene delivery vectors may be derived from different retroviruses.

A retroviral vector, suitable for the expression of a TSPAN-7 inhibitory antisense molecule, preferably includes at least one transcriptional promoter/enhancer or locus defining element(s), or other elements that control gene expression by other means such as alternate splicing, nuclear RNA export, post-translational modification of messenger, or post-transcriptional modification of protein. Such vector constructs preferably also include a packaging signal, long terminal repeats (LTRs) or portion thereof, and positive and negative strand primer binding sites appropriate to the retrovirus used (if these are not already present in the retroviral vector). Optionally, the retroviral vector may also include a signal that directs polyadenylation, selectable markers such as Neomycin resistance, TK, hygromycin resistance, phleomycin resistance histidinol resistance, or DHFR, as well as one or more restriction sites and a translation termination sequence. Within one aspect of the present invention, retroviral gene delivery vector constructs are provided comprising a 5′ LTR, a tRNA binding site, a packaging signal, one or more heterologous sequences, an origin of second strand DNA synthesis and a 3′ LTR, wherein the vector construct lacks gag/pol or env coding sequences.

Other retroviral gene delivery vectors may likewise be utilized within the context of the present invention, including, for example, those disclosed in the following each of which is incorporated herein by reference: EP 0,415,731; WO 90/07936; WO 94/03622; WO 93/25698; WO 93/25234; U.S. Pat. No. 5,219,740; WO 93/11230; WO 93/10218; Vile et al., Cancer Res. 53:3860-3864 (1993); Vile et al., Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127, GB 2,200,651, EP 0,345,242 and WO 91/02805.

Packaging cell lines suitable for use with the above described retroviral gene delivery vector constructs may be readily prepared. See, e.g., U.S. Pat. Nos. 5,716,832 and 5,591,624. These packaging cell lines may be utilized to create producer cell lines (also termed vector cell lines or “VCLs”) for the production of recombinant vector particles. It may be preferred to use packaging cell lines made from human (e.g., HT1080 cells) or mink parent cell lines, thereby allowing production of recombinant retroviruses that avoid inactivation in human serum.

2. Adeno-Associated Viral Gene Delivery Vector Systems

Adeno-associated viruses (AAV) possess a number of qualities that make them particularly suitable for the development of gene delivery vectors generally and for the delivery of polynucleotides encoding TSPAN-7 inhibitory antisense molecules in particular. For a general review of AAV expression systems, see Rabinowitz et al., Current Opin. Biotech. 9(5):470-475 (1998). AAV is a non-pathogenic, defective human parvovirus that is non-infective without an adeno or herpes helper virus. Thus, in the absence of a helper virus, AAV becomes integrated latently into the host genome. In addition, AAV has the advantage over the retroviruses, discussed above, in being able to transduce a wide range of both dividing and quiescent cell types.

A variety of AAV gene delivery vectors may be utilized to direct the expression of one or more TSPAN-7 inhibitor antisense molecule. Representative examples of such vectors include the AAV vectors disclosed by Srivastava in WO 93/09239; Samulski, et al. J. Virol. 63:3822-3828 (1989); Mendelson, et al. Virol. 166:154-165 (1988); and Flotte, et al. Proc. Natl. Acad. Sci. U.S.A. 90(22):10613-10617 (1993) incorporated herein by reference.

Briefly, an AAV gene delivery vector of the present invention may include, in order, a 5′ adeno-associated virus inverted terminal repeat; a polynucleotide encoding the TSPAN-7 inhibitory antisense molecule; a sequence operably linked to the TSPAN-7 inhibitory antisense molecule that regulates its expression in a target tissue, organ or cell; and a 3′ adeno-associated virus inverted terminal repeat. A suitable regulatory sequence for the expression of TSPAN-7 inhibitory antisense molecule is, e.g., the enhancer/promoter sequence of cytomegalovirus (CMV). In addition, the AAV vector may preferably have a polyadenylation sequence such as the bovine growth hormone (BGH) polyadenylation sequence.

Generally, AAV vectors should have one copy of the AAV ITR at each end of the TSPAN-7 inhibitory antisense molecule, to allow replication, packaging, efficient integration into the host cell genome and rescue from the chromosome. The 5′ ITR sequence consists of nucleotides 1 to 145 at the 5′ end of the AAV DNA genome, and the 3′ ITR includes nucleotides 4681 to 4536 of the AAV genome. Preferably, the AAV vector may also include at least 10 nucleotides following the end of the ITR (i.e., a portion of the so-called “D region”).

Optimal packaging of an adeno-associated virus gene delivery vector requires that the 5′ and 3′ ITRs be separated by approximately 2-5 kb. It will be apparent, however, that the ideal spacing between ITR sequences may vary depending on the particular packaging system utilized. This spacing may be achieved by incorporating a “stuffer” or “filler” polynucleotide fragment to bring the total size of the nucleic acid sequence between the two ITRs to between 2 and 5 kb. Thus, where the TSPAN-7 inhibitory antisense molecule is smaller than 2-5 kb, a non-coding stuffer polynucleotide may be incorporated, for example, 3′ to the 5′ ITR sequence and 5′ of the TSPAN-7 inhibitory antisense molecule. The precise nucleotide sequence of the stuffer fragment is not an essential element of the final construct.

Depending upon the precise application contemplated, rather than incorporating a stuffer fragment, multiple copies of the TSPAN-7 inhibitory antisense molecule may be inserted, inter alia, to achieve the optimal ITR sequence spacing. It may be preferred to organize the polynucleotides as two or more separate transcription units each with its own promoter and polyadenylation signal.

Recombinant AAV vectors of the present invention may be generated from a variety of adeno-associated viruses, including for example, serotypes 1 through 6. For example, ITRs from any AAV serotype are expected to have similar structures and functions with regard to replication, integration, excision and transcriptional mechanisms.

Within certain embodiments of the invention, expression of the TSPAN-7 inhibitory antisense molecule may be accomplished by a separate promoter (e.g., a viral promoter). Representative examples of suitable promoters in this regard include a CMV promoter, an RSV promoter, an SV40 promoter, or a MoMLV promoter. Other promoters that may similarly be utilized within the context of the present invention include cell or tissue specific promoters or inducible promoters. Representative inducible promoters include tetracycline-response promoters (e.g., the “Tet” promoter) as described in Gossen et al., Proc. Natl. Acad. Sci. U.S.A. 89:5547-5551 (1992); Gossen et al., Science 268:1766-1769 (1995); Baron et al., Nucl. Acids Res. 25:2723-2729 (1997); Blau et al., Proc. Natl. Acad. Sci. USA. 96:797-799 (1999); Bohl et al., Blood 92:1512-1517 (1998); and Haberman et al., Gene Therapy 5:1604-1611 (1998); the ecdysone promoter system as described in No et al., Proc. Natl. Acad. Sci. USA. 93:3346-3351 (1996); and other regulated promoters or promoter systems as described in Rivera et al., Nat. Med. 2:1028-1032 (1996).

The AAV gene delivery vector may also contain additional sequences, for example from an adenovirus, which assist in effecting a desired function for the vector. Such sequences include, for example, those which assist in packaging the AAV gene delivery vector in adenovirus particles.

Packaging cell lines suitable for producing adeno-associated viral vectors may be routinely prepared given readily available techniques. See, e.g., U.S. Pat. No. 5,872,005, incorporated herein by reference. At a minimum, suitable packaging systems for AAV gene delivery systems of the present invention will include the AAV replication and capsid genes.

Preferred packaging cell lines may contain both an AAV helper virus as well as an AAV gene delivery vector containing the TSPAN-7 inhibitory antisense molecule. For detailed descriptions of representative packaging cell line systems, see, e.g., Holscher, C. et al., J. Virol. 68:7169-7177 (1994); Clark, K. R. et al., Hum. Gene Ther. 6:1329-1341 (1995); and Tamayosa, K. et al., Hum. Gen. Ther 7:507-513 (1996) which are incorporated herein by reference.

Alternatively, packaging of AAV may be achieved in vitro in a cell free system to obviate transfection protocols or packaging cell lines. Such in vitro systems incorporate an AAV gene delivery vector bearing the TSPAN-7 inhibitory antisense molecule and a source of Rep-protein, capsid-protein and Adenovirus proteins that supply helper-viral functions. The latter proteins are typically supplied in the form of a cell extract. Representative in vitro systems are further described in Ding, L. et al., Gen. Ther. 4:1167-1172 (1997) and Zhou, Z. et al., J. Virol. 72:3241-3247 (1998) which are incorporated herein by reference.

3. Other Viral Gene Delivery Vector Systems

In addition to retroviral vectors and adeno-associated virus-based vectors, numerous other viral gene delivery vector systems may also be utilized for the expression of TSPAN-7 inhibitory antisense molecules. For example, within one embodiment of the invention adenoviral vectors may be employed. Representative examples of such vectors include those described by, for example, Berkner, Biotechniques 6:616-627 (1988); Rosenfeld et al., Science 252:431-434 (1991); WO 93/9191; Kolls et al., Proc. Natl. Acad. Sci. U.S.A. 91(1):215-219 (1994); Kass-Eisler et al., Proc. Natl. Acad. Sci. U.S.A. 90(24):11498-502 (1993); Guzman et al., Circulation 88(6):2838-48 (1993); Guzman et al., Cir. Res. 73(6):1202-1207 (1993); Zabner et al., Cell 75(2):207-216 (1993); Li et al. Hum. Gene Ther. 4(4):403-409 (1993); Caillaud et al., Eur. J. Neurosci. 5(10):1287-1291 (1993); Vincent et al., Nat. Genet. 5(2):130-134 (1993); Jaffe et al., Nat. Genet. 1(5):372-378 (1992); and Levrero et al., Gene 101(2):195-202 (1991); and WO 93/07283; WO 93/06223; and WO 93/07282.

Gene delivery vectors of the present invention also include herpes vectors. Representative examples of such vectors include those disclosed by Kit in Adv. Exp. Med. Biol. 215:219-236 (1989); and those disclosed in U.S. Pat. No. 5,288,641 and EP 0176170 (Roizman). Additional exemplary herpes simplex virus vectors include HFEM/ICP6-LacZ disclosed in WO 95/04139 (Wistar Institute), pHSVlac described in Geller, Science 241:1667-1669 (1988), and in WO 90/09441 and WO 92/07945; HSV Us3::pgC-lacZ described in Fink, Human Gene Therapy 3:11-19 (1992); and HSV 7134, 2 RH 105 and GAL4 described in EP 0453242 (Breakefield), and those deposited with the ATCC as accession numbers ATCC VR-977 and ATCC VR-260.

Gene delivery vectors may also be generated from a wide variety of other viruses including, for example, poliovirus (Evans et al., Nature 339:385-388 (1989); and Sabin, J. Biol. Standardization 1:115-118 (1973)); rhinovirus; pox viruses, such as canary pox virus or vaccinia virus (Fisher-Hoch et al., Proc. Natl. Acad. Sci. U.S.A. 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330 and 5,017,487; WO 89/01973); SV40 (Mulligan et al., Nature 277:108-114 (1979); influenza virus (Luytjes et al., Cell 59:1107-1113 (1989); McMicheal et al., N. Eng. J Med. 309:13-17 (1983); and Yap et al., Nature 273:238-239 (1978)); HIV (Poznansky, J. Virol. 65:532-536 (1991)); measles (EP 0 440,219); astrovirus (Munroe et al., J. Vir. 67:3611-3614 (1993)); and coronavirus, as well as other viral systems (e.g., EP 0,440,219; WO 92/06693; U.S. Pat. No. 5,166,057).

4. Non-viral Gene Delivery Vectors

Other gene delivery vectors and methods may be employed for the expression of TSPAN-7 inhibitory antisense molecules such as, for example, nucleic acid expression vectors; polycationic condensed DNA linked or unlinked to killed adenovirus alone, for example, see Curiel, Hum Gene Ther 3:147-154 (1992); ligand linked DNA, for example, see Wu, J. Biol Chem 264:16985-16987 (1989); eucaryotic cell delivery vectors; deposition of photopolymerized hydrogel materials; hand-held gene delivery particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol Cell Biol 14:2411-2418 (1994), and in Woffendin, Proc. Natl. Acad. Sci. 91:1581-1585 (1994).

Particle mediated gene delivery may be employed. Briefly, the TSPAN-7 inhibitory antisense molecule of interest can be inserted into conventional vectors that contain conventional control sequences for high level expression, and then be incubated with synthetic gene delivery molecules such as polymeric DNA-binding cations like polylysine, protamine, and albumin, linked to cell targeting ligands such as asialoorosomucoid, as described in Wu, et al., J. Biol. Chem. 262:4429-4432 (1987), insulin as described in Hucked, Biochem Pharmacol 40:253-263 (1990), galactose as described in Plank, Bioconjugate Chem 3:533-539 (1992), lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, PCT Patent Publication Nos. WO 95/13796, WO 94/23697, and WO 91/144445, and European Patent Publication No. 524,968. Nucleic acid sequences can be inserted into conventional vectors that contain conventional control sequences for high level expression, and then be incubated with synthetic gene delivery molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al., Proc. Natl. Acad. Sci. U.S.A. 91 (24):11581-11585 (1994). Moreover, the coding sequence and the product of expression of such can be delivered through deposition of photopolymerized hydrogel materials.

Exemplary liposome and polycationic gene delivery vehicles are those described in U.S. Pat. Nos. 5,422,120 and 4,762,915, in PCT Patent Publication Nos. WO 95/13796, WO 94/23697, and WO 91/14445, in European Patent Publication No. 524,968 and in Starrier, Biochemistry, pp. 236-240 (1975) W. H. Freeman, San Francisco; Shokai, Biochem. Biophys. Acta. 600:1 (1980); Bayer, Biochem. Biophys. Acta. 550:464 (1979); Rivet, Methods Enzymol. 149:119 (1987); Wang, Proc. Natl. Acad. Sci. U.S.A. 84:7851 (1987); Plant, Anal. Biochem. 176:420 (1989). Exemplary lipitoid carriers are disclosed in W098/06437, and WO01/16306 (with reference to antisense molecules), and exemplary cholesteroid carriers are disclosed in W099/08711, all of which are incorporated by reference herein.

### EXAMPLES

The following experimental examples are offered by way of illustration, not limitation.

### Example 1

### Antisense Inhibition of TSPAN-7 mRNA

A. Preparation of Transfection Mixture

For each transfection mixture, a carrier molecule, preferably a lipitoid or cholesteroid, was prepared to a working concentration of 0.5 mM in water, sonicated to yield a uniform solution, and filtered through a 0.45 μm PVDF membrane. The antisense or control oligonucleotide (FIG. 4, SEQ ID NO:3-12) was prepared to a working concentration of 100 μM in sterile Millipore water.

The oligonucleotide was diluted in OptiMEM™ (Gibco/BRL), in a microfuge tube, to 2 μM, or approximately 20 μg oligo/ml of OptiMEM™. In a separate microfuge tube, lipitoid or cholesteroid, typically in the amount of about 1.5-2 nmol lipitoid/μg antisense oligonucleotide, was diluted into the same volume of OptiMEM™ used to dilute the oligonucleotide. The diluted antisense oligonucleotide was immediately added to the diluted lipitoid and mixed by pipetting up and down.

B. Transfection

SW620 cells were plated on tissue culture dishes one day in advance of transfection, in growth media with serum, to yield a density at transfection of 60-90%. The oligonucleotide/lipitoid mixture was added to the cells, immediately after mixing, to a final concentration of 100-300 nM antisense oligonucleotide. Cells were incubated with the transfection mixture at 37° C., 5% CO₂ for 4-24 hours. After incubation, the transfection mixture was removed and replaced with normal growth media with serum.

Total RNA was extracted using the RNeasy™ kit (Quiagen Corporation, Chatsworth, Calif.), according to manufacturer's protocols.

C. Reverse Transcription

The level of target mRNA was quantitated using the Roche LightCycler™ real-time PCR machine. Values for the target mRNA were normalized versus an internal control (e.g., beta-actin).

For each 20 μl reaction, extracted RNA (generally 0.2-1 μg total) was placed into a sterile 0.5 or 1.5 ml microcentrifuge tube, and water was added to a total volume of 12.5 μl. To each tube was added 7.5 μl of a buffer/enzyme mixture, prepared by mixing (in the order listed) 2.5 μl H₂O, 2.0 μl 10× reaction buffer, 10 μl oligo dT (20 pmol), 1.0 μl dNTP mix (10 mM each), 0.5 μl RNAsin® (20u) (Ambion, Inc., Hialeah, Fla.), and 0.5 μl MMLV reverse transcriptase (50u) (Ambion, Inc.). The contents were mixed by pipetting up and down, and the reaction mixture was incubated at 42° C. for I hour. The contents of each tube were centrifuged prior to amplification.

D. LightCycler m Amplification of RT Reactions

An amplification mixture was prepared by mixing in the following order: 1× PCR buffer II, 3 mM MgCl₂, 140 μM each dNTP, 0.175 pmol each oligo, 1:50,000 dil of SYBR® Green, 0.25 mg/ml BSA, 1 unit Taq polymerase, and H₂O to 20 μl. (PCR buffer II is available in 10× concentration from Perkin-Elmer, Norwalk, Conn.). In 1× concentration it contains 10 mM Tris pH 8.3 and 50 mM KCl. SYBR® Green (Molecular Probes, Eugene, Oreg.) is a dye which fluoresces when bound to double stranded DNA. As double stranded PCR product is produced during amplification, the fluorescence from SYBR® Green increases.

To each 20 μl aliquot of amplification mixture, 2 μl of template RT was added, and amplification was carried out according to standard protocols.

As shown in FIG. 5 and in Table 1 below, TSPAN-7 message levels were decreased relative to actin in SW620 cells.

TABLE 1
Effect of TSPAN-7 Oligonucleotides on SW620 Proliferation

| | TSPAN-7 message levels |
|---|---|
| Antisense oligonucleotide | normalized to actin |
| 22-1 AS | 0.21 |
| SEQ ID NO:2 | |
| 22-2 AS | 0.17 |
| SEQ ID NO:3 | |
| 22-3 AS | 0.16 |
| SEQ ID NO:4 | |
| 22-4 AS | 0.14 |
| SEQ ID NO:5 | |
| 22-5 AS | 0.11 |
| SEQ ID NO:6 | |
| 22-1 RC | 0.4 |
| SEQ ID NO:7 | |
| 22-2 RC | 0.36 |
| SEQ ID NO:8 | |
| 22-3 RC | 0.15 |
| SEQ ID NO:9 | |
| 22-4 RC | 0.51 |
| SEQ ID NO:10 | |
| 22-5 RC | 0.49 |
| SEQ ID NO:11 | |

### Example 2

### Cell Proliferation Assay

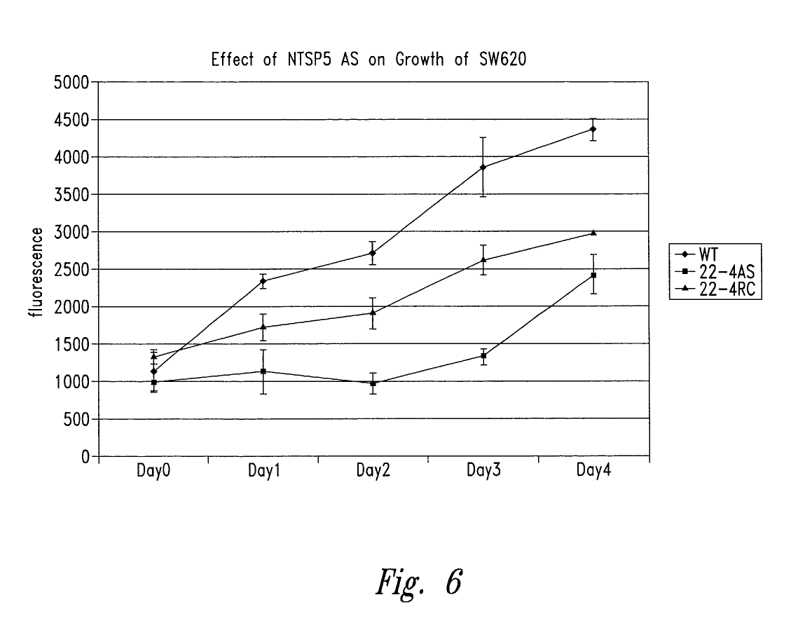

Cells were seeded into 96 well plates at a density of 5000 cells per well. For a 4 day proliferation assay, 5 independent 96 well plates were prepared, one for each day. After overnight incubation, cells were transfected using the procedure described above. On each day of the proliferation assay, all medium was removed from one plate and frozen at −70° C. On day four, all plates were developed with the Quantos™ assay kit (Stratagene, La Jolla, Calif.) which determines the amount of DNA, and thus the number of cells, in each well. The results are shown in FIG. 6 and Table 2 below.

TABLE 2
Effect of TSPAN-7 Oligonucleotides on Growth of SW620 Cells

| Oligonucleotide | Day 0 | Day 1 | Day 2 | Day 3 | Day 4 |
|---|---|---|---|---|---|
| Wild type (no oligo) | 1200 | 2300 | 2700 | 3800 | 4250 |
| 22-4AS | 1000 | 1000 | 1000 | 1300 | 2300 |
| 22-4RC | 1300 | 1700 | 1900 | 2500 | 3000 |

From the foregoing it will be appreciated that, although specific embodiments of the invention have been described herein for purposes of illustration, various modifications may be made without departing from the spirit and scope of the invention. Accordingly, the invention is not limited except as by the appended claims.

What is claimed is:

1. An isolated TSPAN-7 inhibitor wherein said TSPAN-7 inhibitor is selected from the group consisting of an antisense molecule, an antibody, a ribozyme, a peptide, a peptide mimetic, and a cyclic peptide.

2. The isolated TSPAN-7 inhibitor of claim 1 wherein said antisense molecule or the complement thereof comprises an oligonucleotide selected from the group consisting of at least 10, at least 16, at least 20, and at least 25 consecutive nucleotides of the sequence of SEQ ID NO:1.

3. The isolated TSPAN-7 inhibitor of claim 1 wherein said antisense molecule or the complement thereof hybridizes under high stringency conditions to the sequence of SEQ ID NO:1.

4. The isolated TSPAN-7 inhibitor of claim 1 wherein said antisense molecule comprises a nucleic acid sequence selected from the group consisting of SEQ ID NO:3-7.

5. An isolated TSPAN-7 inhibitor wherein said TSPAN-7 inhibitor is a ribozyme.

6. The isolated TSPAN-7 inhibitor of claim 1, wherein said inhibitor is an antibody selected from the group consisting of a monoclonal antibody, a polyclonal antibody, a humanized antibody, a human antibody, an antibody fragment, a bispecific antibody, and a trispecific antibody.

7. The isolated TSPAN-7 inhibitor of claim 6, wherein said antibody fragment is selected from the group consisting of a F(ab′)₂ and a single chain Fv fragment.

8. A method of decreasing the expression of TSPAN-7 in a mammalian cell, comprising administering to said cell a TSPAN-7 inhibitor of claim 1.

9. The method of claim 6 wherein said TSPAN-7 inhibitor is an antisense molecule comprising an oligonucleotide selected from the group consisting of at least 10, at least 16, at least 20, and at least 25 consecutive nucleic acids of the sequence of SEQ ID NO:1.

10. A method of treating hyperproliferative disorder, comprising administering to a mammalian cell a TSPAN-7 inhibitor such that said hyperproliferative disorder is reduced in severity.

11. The method of claim 8 wherein said hyperproliferative disorder is cancer.

12. A method of making a recombinant vector comprising inserting an oligonucleotide selected from the group consisting of at least 10, at least 16, at least 20, and at least 25 consecutive nucleotides of SEQ ID NO:1, or the complement thereof, into a vector in operable linkage to a promoter.

13. A recombinant vector produced by the method of claim 12.

14. A method of making a recombinant host cell comprising introducing the recombinant vector of claim 13 into a host cell.

15. A recombinant host cell produced by the method of claim 14.

16. A recombinant method of producing a polypeptide, comprising culturing the recombinant host cell of claim 15 under conditions such that said polypeptide is expressed and recovering said polypeptide.

17. An epitope-bearing portion of the polypeptide of SEQ ID NO:2.

18. The epitope-bearing portion of claim 17, which comprises about 8 to about 25 contiguous amino acids of any one of SEQ ID NO:2, SEQ ID NO:13 and SEQ ID NO:14.

19. The epitope-bearing portion of claim 17, which comprises about 10 to about 15 contiguous amino acids of SEQ ID NO:2, SEQ ID NO:13 and SEQ ID NO:14.

20. An isolated antibody that binds specifically to a polypeptide comprising amino acids at least 95% identical to a polypeptide comprising amino acids from about I to about 270 of SEQ ID NO:2.

21. An isolated antibody that binds specifically to an isolated polypeptide wherein. except for at least one conservative amino acid substitution, said polypeptide has an amino acid sequence selected from the group consisting of:
(a) amino acids from about 1 to about 270 of SEQ ID NO:2; and
(b) amino acids from about 2 to about 270 of SEQ ID NO:2.

22. An isolated antibody that binds specifically to a polypeptide comprising the epitope-bearing portion of claim 17.


SEQUENCE LISTING (14 sequences)

SEQ ID NO: 1 - DNA, 1388 bp, Homo sapiens
  misc_feature 1285, 1377: n = A,T,C or G
cttcctcggccgagccgggccgcgcggccgctgccgccgccgcgcgcggattctgcttctcagaagatgcactattatagatactctaacgccaaagtcagctgctggtacaagtacctccttttcagctacaacatcatcttctggttggctggagttgtcttccttggagtcgggctgtgggcatggagcgaaaagggtgtgctgtccgacctcaccaaagtgacccggatgcatggaatcgaccctgtggtgctggtcctgatggtgggcgtggtgatgttcaccctggggttcgccggctgcgtgggggctctgcgggagaatatctgcttgctcaactttttctgtggcaccatcgtgctcatcttcttcctggagctggctgtggccgtgctggccttcctgttccaggactgggtgagggaccggttccgggagttcttcgagagcaacatcaagtcctaccgggacgatatcgatctgcaaaacctcatcgactcccttcagaaagctaaccagtgctgtggcgcatatggccctgaagactgggacctcaacgtctacttcaattgcagcggtgccagctacagccgagagaagtgcggggtccccttctcctgctgcgtgccagatcctgcgcaaaaagttgtgaacacacagtgtggatatgatgtcaggattcagctgaagagcaagtgggatgagtccatcttcacgaaaggctgcatccaggcgctggaaagctggctcccgcggaacatttacattgtggctggcgtcttcatcgccatctcgctgttgcagatatttggcatcttcctggcaaggacgctgatctcagacatcgaggcagtgaaggccggccatcacttctgaggagcagagttgagggagccgagctgagccacgctgggaggccagagcctttctctgccatcagccctacgtccagagggagaggagccgacacccccagagccagtgccccatcttaagcatcagcgtgacgtgacctctctgtttctgcttgctggtgctgaagaccaagggtcccccttgttacctgcccaaacttgtgactgcatccctctggagtctacccagagacagagaatgtgtctttatgtgggagtggtgactctgaaagacagagagggctcctgtggctgccaggagggcttgactcagaccccctgcagctcaagcatgtctgcaggacaccctggtccccctctccaytggcwtccagacatctgctttgggtcatccacatctgtgggtnggccgtgggtagagggacccacaggcgtggacagggcatctctctccatcaagcaaagcagcatggggggccttgccgtaaacgggaggcgngacgttggccc

SEQ ID NO: 2 - protein, 270 aa, Homo sapiens
MHYYRYSNAKVSCWYKYLLFSYNIIFWLAGVVFLGVGLWAWSEKGVLSDLTKVTRMHGIDPVVLVLMVGVVMFTLGFAGCVGALRENICLLNFFCGTIVLIFFLELAVAVLAFLFQDWVRDRFREFFESNIKSYRDDIDLQNLIDSLQKANQCCGAYGPEDWDLNVYFNCSGASYSREKCGVPFSCCVPDPAQKVVNTQCGYDVRIQLKSKWDESIFTKGCIQALESWLPRNIYIVAGVFIAISLLQIFGIFLARTLISDIEAVKAGHHF

SEQ ID NO: 3 - DNA, 25 bp, Artificial Sequence
  Oligonucletoide sequence
tgcagcctttcgtgaagatggactc

SEQ ID NO: 4 - DNA, 25 bp, Artificial Sequence
  Oligonucletoide sequence
ccccatgctgctttgcttgatggag

SEQ ID NO: 5 - DNA, 23 bp, Artificial Sequence
  Oligonucletoide sequence
gctcagctcggctccctcaactc

SEQ ID NO: 6 - DNA, 25 bp, Artificial Sequence
  Oligonucletoide sequence
cacaagtttgggcaggtaacaaggg

SEQ ID NO: 7 - DNA, 25 bp, Artificial Sequence
  Oligonucletoide sequence
agaggtcacgtcacgctgatgctta

SEQ ID NO: 8 - DNA, 25 bp, Artificial Sequence
  Oligonucletoide sequence
ctcaggtagaagtgctttccgacgt

SEQ ID NO: 9 - DNA, 25 bp, Artificial Sequence
  Oligonucletoide sequence
gaggtagttcgtttcgtcgtacccc

SEQ ID NO: 10 - DNA, 23 bp, Artificial Sequence
  Oligonucletoide sequence
ctcaactccctcggctcgactcg

SEQ ID NO: 11 - DNA, 25 bp, Artificial Sequence
  Oligonucletoide sequence
gggaacaatggacgggtttgaacac

SEQ ID NO: 12 - DNA, 25 bp, Artificial Sequence
  Oligonucletoide sequence
attcgtagtcgcactacgctggaga

SEQ ID NO: 13 - protein, 24 aa, Homo sapiens
AWSEKGVLSDLTKVTRMHGIDPVV

SEQ ID NO: 14 - protein, 120 aa, Homo sapiens
FLFQDWVRDRFREFFESNIKSYRDDIDLQNLIDSLQKANQCCGAYGPEDWDLNVYFNCSGASYSREKCGVPFSCCVPDPAQKVVNTQCGYDVRIQLKSKWDESIFTKGCIQALESWLPRN